(19) United States
(12) Patent Application Publication
(10) Pub. No.: US 2024/0156718 A1
(43) Pub. Date: May 16, 2024

(54) TOPICAL COMPOSITION AND USE THEREOF

(71) Applicant: BLIS TECHNOLOGIES LIMITED, Dunedin (NZ)

(72) Inventors: John David Francis HALE, Dunedin (NZ); Rohit JAIN, Dunedin (NZ); Abigail Louise VOSS, Dunedin (NZ)

(21) Appl. No.: 18/548,143

(22) PCT Filed: Mar. 3, 2022
(86) PCT No.: PCT/IB2022/051860

§ 371 Date: Aug. 28, 2023

(30) Foreign Application Priority Data
Mar. 4, 2021 (AU) 2021900605

(51) Int. Cl. A61K 8/99; A61K 8/03; A61K 8/25; A61K 8/37; A61K 8/60; A61K 8/67; A61K 8/92; A61Q 19/08

(52) U.S. Cl. CPC A61K 8/99; A61K 8/03; A61K 8/25; A61K 8/375; A61K 8/608; A61K 8/678; A61K 8/92; A61Q 19/08

(57) ABSTRACT

Described herein are methods of improving the appearance of skin, or at least one sign of skin aging, using Micrococcus luteus compositions. The invention also relates to topical compositions, kits useful in such methods, and to methods of manufacturing topical compositions.

### FIELD OF THE INVENTION

The present invention relates to methods of improving the appearance of skin, or at least one sign of skin aging, using Micrococcus luteus compositions. The invention also relates to topical compositions and kits useful in such methods.

### BACKGROUND TO THE INVENTION

Skin-care products containing probiotic microorganisms are becoming increasingly well-known. The microorganisms or related products used in skin-care products to date are generally Bifidobacterium spp, Lactobacillus spp (now known as, Limosilactobacillus spp., Lacticaseibacillus spp., Lactiplantibacillus spp., and Ligilactobacillus spp.), Lactococcus spp, and Streptococcus spp, or filtrates or lysates created from the bacteria. However, the role they play in interacting with the skin microbiome is not well understood.

WO2006104403 (Blis Technologies Limited) describes Micrococcus luteus (M. luteus) compositions and their therapeutic use for controlling skin diseases or disorders. Probiotic strain Q24 on deposit at Deutsche Sammlung von Mikro organisms Und Zellkulturen GmbH, Braunschweig, Germany, under accession number DSM 17172 is also provided. This document is incorporated herein by reference in its entirety.

ANZCTR, A Probiotic for Eczema Treatment (Registration number: ACTRN12616000022460) describes a clinical trial to test the use of a lysate of Micrococcus luteus Q24 for the treatment of eczema. No results are provided.

The applicants have unexpectedly identified a new role for M. luteus in cosmetic applications. Such cosmetic applications include improving skin appearance, or at least one sign of skin aging. Based on this unexpected finding, there is a need to develop new cosmetic treatment methods and compositions suitable for cosmetic use.

Working with probiotic organisms is challenging. Probiotics are sensitive and often respond unpredictably to a range of freeze-drying processes, agents used in such processes, processing conditions and formulation agents, amongst others.

In the harsh freeze-drying processes, microorganisms are generally protected using a range of lyoprotectants and cryoprotectants. The applicants have also surprisingly found that M. luteus can be formulated in the absence of a lyoprotectant or cryoprotectant without significant loss in viability of the probiotic microorganism.

Preparing formulations comprising Micrococcus luteus can be challenging as M. luteus has been found to be unstable in aqueous and polar solvents, and is chemical and heat sensitive.

It is an object of the invention to provide methods for improving the appearance of skin, or at least one sign of skin aging using Micrococcus luteus compositions; and/or to at least provide the public with a useful choice.

Other objects of the invention may become apparent from the following description which is given by way of example only.

Any discussion of documents, acts, materials, devices, articles, or the like which has been included in the present specification is solely for the purpose of providing a context for the present invention. It is not to be taken as an admission that any or all of these matters form part of the prior art base or were common general knowledge in the field relevant to the present invention as it existed before the priority date.

### SUMMARY OF THE INVENTION

In a first aspect, the present invention relates to a method to improve appearance of skin or at least one sign of aging comprising applying to the skin a topical composition comprising Micrococcus luteus Q24.

In a second aspect, the invention provides a topical composition comprising Micrococcus luteus Q24, a viscosity modifier, a dispersing agent and an oil vehicle, wherein the composition comprises Micrococcus luteus Q24 in an amount of about 1×10⁴ to about 1×10¹⁰ cfu/g.

In a third aspect, the invention provides a topical composition comprising Micrococcus luteus Q24, hydrophobic silica, polysorbate 80, and an oil vehicle.

In a fourth aspect, the invention provides a topical composition comprising
about 1×10³ to about 1×10¹² cfu/g Micrococcus luteus Q24,
about 2 to about 10% w/w hydrophobic silica,
about 0.5 to about 2% w/w polysorbate 80, and
a quantity sufficient amount of oil vehicle.

In a fifth aspect, the invention provides a topical composition as defined in any one of the first to fourth aspects for improving the appearance of skin or at least one sign of aging.

In a sixth aspect, the invention relates to use of Micrococcus luteus Q24 in the manufacture of medicament for improving the appearance of skin or at least one sign of aging.

In a seventh aspect, the invention provides a two-phase composition comprising an oil phase and an aqueous phase, wherein the oil phase comprises a topical composition according to any one of the second to fourth aspects.

In an eighth aspect, the invention provides a kit comprising a topical composition comprising Micrococcus luteus Q24 and an aqueous composition.

In a ninth aspect, the invention relates to a method of manufacturing a topical composition comprising Micrococcus luteus Q24 comprising the steps of:
a) mixing an oil vehicle and dispersing agent,
b) adding Micrococcus luteus Q24 and a viscosity modifier to the mixture from step a),
c) homogenising the mixture from step b) to provide the composition.

The following embodiments and preferences may relate alone or in any combination of any two or more to any of the above aspects.

In various embodiments, the composition comprises Micrococcus luteus Q24 in an amount of about 1×10³ to about 1×10¹² cfu/g.

In various embodiments, the composition comprises a viscosity modifier.

In various embodiments, the composition comprises the viscosity modifier in an amount of about 3 to about 15% w/w.

In various embodiments, the viscosity modifier is selected from the group consisting of hydrophobic silica, hydrophilic silica, white beeswax, yellow beeswax, paraffin wax, jojoba wax, microcrystalline wax, ethyl cellulose, stearic acid, xanthan gum, tapioca starch, Carbopol polymer, cocoa butter, rhea butter, and a combination of any two or more thereof.

In various embodiments, the composition comprises a dispersing agent.

In various embodiments, the composition comprises the dispersing agent in an amount of about 0.1 to about 5% w/w.

In various embodiments, the dispersing agent is selected from the group consisting of polysorbate 80, polysorbate 20, sorbitan oleate, egg lecithin, soybean lecithin, polyoxyl 35 castor oil, and a combination of any two or more thereof.

In various embodiments, the composition comprises an oil vehicle.

In various embodiments, the oil vehicle is selected from the group consisting of a medium chain triglyceride, plant oil, or a combination thereof.

In various embodiments, the medium chain triglyceride is a caprylic/capric triglyceride.

In various embodiments, the plant oil is selected from the group consisting of sunflower oil, canola oil, soybean oil, olive oil, jojoba oil, argan oil, rosehip oil, marula oil, chamomile oil, tamanu oil, grapeseed oil, and a combination of any two or more thereof.

In various embodiments, Micrococcus luteus Q24 is lyoprotectant-free.

In various embodiments, the composition further comprises one or more additional probiotics.

In various embodiments, the one or more additional probiotic is selected from the group consisting of a Streptococcus spp., a Lactobacillus spp., Limosilactobacillus spp., a Lacticaseibacillus spp., a Ligilactobacillus spp., Lactiplantibacillus spp., a Bifidobacterium spp., a Saccharomyces spp., and a combination of any two or more thereof.

In various embodiments, the Streptococcus spp. is selected from the group consisting of Streptococcus salivarius K12, Streptococcus salivarius M18, Streptococcus salivarius 24 SMB, Streptococcus oralis (e.g. S. oralis 89a), and a combination of any two or more thereof. In various embodiments, the Streptococcus spp. is selected from the group consisting of Streptococcus salivarius K12, Streptococcus salivarius M18, Streptococcus salivarius 24 SMB, Streptococcus oralis (e.g. S. oralis 89a), Streptococcus salivarius DB-B5, and a combination of any two or more thereof.

In various embodiments, the composition comprises each additional probiotic in an amount of about 1×10³ to about 1×10¹² cfu/g.

In various embodiments, the composition further comprises an inhibitory activity enhancer, a buffering agent, an antibacterial agent, a prebiotic, a fragrance, an antioxidant, a colourant, a skin protective agent, an antimicrobial, an aluminium salt, a mineral pigment, an odour absorbant or neutraliser, a sunscreen agent, and a combination of any two or more thereof.

In various embodiments, the inhibitory activity enhancer is selected from the group consisting of sodium chloride, ethylenediaminetetraacetic acid, arginine, calcium carbonate, and a combination of any two or more thereof.

In various embodiments, the buffering agent is selected from the group consisting of calcium carbonate, magnesium carbonate, sodium hydrogen phosphate, sodium dihydrogen phosphate, dipotassium hydrogen phosphate, and potassium dihydrogen phosphate, magnesium carbonate, urea, hydrated aluminium oxides, hydrated aluminum oxides, bentonite clays, kaolin clay, and a combination thereof.

In various embodiments, the antibacterial agent is selected from the group consisting of xylitol, erythritol, epidermin, nisin, salivaricin A, salivaricin A1, salivaricin A2, salivaricin B, and a combination thereof. In various embodiments, the antibacterial agent is selected from the group consisting of xylitol, erythritol, epidermin, nisin, salivaricin A, salivaricin A1, salivaricin A2, salivaricin B, salivaricin 9, salivaricin MPS, and a combination thereof.

In various embodiments, the prebiotic is selected from the group consisting of Manuka honey powder, olive squalene, pomegranate seed oil, flax seed oil, coconut oil, colloidal oatmeal, vitamin E, vitamin C, retinol (vitamin A), olive oil, hyaluronic acid, calendula oil, almond oil, tomato oil, allantoin, aloe vera powder, colloidal oatmeal, xylitol, yeast extract, fructooligosaccharide powder, fructooligosaccharide liquid, fructooligosaccharides, galactooligosaccharides, xylooligosaccharides, niacinamide, sunscreen, and a combination of any two or more thereof.

In various embodiments, the prebiotic is selected from the group consisting of yeast extract, cysteine, maltodextrin, inulin, liquorice root, liquorice extract, honey, and a combination of any two or more thereof.

In various embodiments, the fragrance is selected from the group consisting of rose water, orange blossom, rose gardenia, peony, white jasmine, ylang ylang oil, geranium oil, rose oil, and a combination of any two or more thereof.

In various embodiments, the antioxidant is selected from the group consisting of vitamin E, resveratrol, squalene, vitamin C, β-carotene, retinyl acetate, retinyl palmitate, retinol, niacinamide, green tea, green tea extract, caffeine, and a combination of any two or more thereof.

In various embodiments the antioxidant is selected from olive squalene, pomegranate seed oil, flax seed oil, coconut oil, colloidal oatmeal, vitamin E, vitamin C, retinol (vitamin A), olive oil, hyaluronic acid, calendula oil, almond oil, tomato oil, and a combination of any two or more thereof.

In various embodiments, the skin protective agent is selected from the group consisting of ceramide, collagen, elastin, coenzyme Q10, hydrolysed collagen, hyaluronic acid, sodium hyaluronate, retinol, palmitoyl tripeptide-1, palmitoyl tetrapeptide-7, palmitoyl tetrapeptide-38, acetyl hexapeptide-8, heptapeptide-14, heptapeptide-15-palmitate, palmitoyl tetrapeptide-7, palmitoyl tripeptide-1, alpha-hydroxy acids, beta-hydroxy acids, vitamin B5, seaweed, seaweed extract, silicone, xylitol, oat extract, oatmeal powder, colloidal oatmeal, aloe vera, and a combination of any two or more thereof. In various embodiments, the skin protective agent is selected from the group consisting of ceramide, collagen, elastin, coenzyme Q10, hydrolysed collagen, hyaluronic acid, sodium hyaluronate, retinol, palmitoyl tripeptide-1, palmitoyl tetrapeptide-7, palmitoyl tetrapeptide-38, acetyl hexapeptide-8, heptapeptide-14, heptapeptide-15-palmitate, palmitoyl tetrapeptide-7, palmitoyl tripeptide-1, alpha-hydroxy acids, beta-hydroxy acids, vitamin B5, seaweed, seaweed extract, silicone, xylitol, oat extract, oatmeal powder, colloidal oatmeal, aloe vera, sunscreen, and a combination of any two or more thereof.

In various embodiments, the antimicrobial is selected from the group consisting of zinc, salicylic acid, azelaic acid, benzoyl peroxide, and a combination thereof.

In various embodiments, the composition comprises from about 0.1% to about 10% w/w olive squalene, and from about 0.1 to about 10% w/w pomegranate seed oil.

In various embodiments, the composition comprises from about 0.1% to about 10% w/w olive squalene, and from about 0.1 to about 3% w/w vitamin E.

In various embodiments, the composition comprises from about 0.1% to about 10% w/w olive squalene, and from about 0.1 to about 10% w/w pomegranate seed oil, and from about 0.1 to about 3% w/w vitamin E.

In various embodiments, improving the appearance of skin or at least one sign of aging includes that skin looks more radiant, skin looks healthier, skin feels more hydrated, pores are reduced in size, skin feels softer, skin looks clearer, wrinkles are reduced, dryness is reduced, spots are reduced, impurities are reduced, moisture is increased, and sebum production is decreased.

In various embodiments, the composition is non-aqueous.

In various embodiments, when applied, the composition is applied in combination with an aqueous phase.

In various embodiments, the composition has a viscosity of from about 20,000 to about 500,000 cp at 25° C. In various embodiments, the composition has a viscosity of from about 20,000 to about 2,000,000 cp at 25° C.

In various embodiments, the composition has a shelf-life of at least 6 months at 25° C. at 60% RH.

In various embodiments, the oil vehicle is a medium chain triglyceride.

In various embodiments, the composition further comprises 0.1 to 10% w/w prebiotic(s). In various embodiments, the composition further comprises 0.1 to 35% w/w prebiotic(s).

In various embodiments, the prebiotic(s) is selected from olive squalene, pomegranate seed oil, vitamin E, or a combination thereof.

In various embodiments, the prebiotics are selected from a combination of olive squalene and pomegranate seed oil; olive squalene and vitamin E; and olive squalene and pomegranate seed oil and vitamin E.

In various embodiments, the kit comprises a dispensing system having a first reservoir and a second reservoir, wherein the first reservoir comprises an oil phase comprising Micrococcus luteus Q24 and the second reservoir comprises an aqueous phase.

In various embodiments, the particle size (Dv90) of Micrococcus luteus Q24 is less than about 300 μm.

In various embodiments, the particle size of Micrococcus luteus Q24 is less than about 250 μm, or less than about 100 μm.

In various embodiments, the method is carried out without heating.

The invention may also be said broadly to consist in the parts, elements and features referred to or indicated in the specification of the application, individually or collectively, in any or all combinations of two or more of said parts, elements or features, and where specific integers are mentioned herein which have known equivalents in the art to which the invention relates, such known equivalents are deemed to be incorporated herein as if individually set forth.

It is intended that reference to a range of numbers disclosed herein (for example, 1 to 10) also incorporates reference to all rational numbers within that range (for example, 1, 1.1, 2, 3, 3.9, 4, 5, 6, 6.5, 7, 8, 9, and 10) and also any range of rational numbers within that range (for example, 2 to 8, 1.5 to 5.5, and 3.1 to 4.7) and, therefore, all sub-ranges of all ranges expressly disclosed herein are hereby expressly disclosed. These are only examples of what is specifically intended and all possible combinations of numerical values between the lowest value and the highest value enumerated are to be considered to be expressly stated in this application in a similar manner.

In this specification where reference has been made to patent specifications, other external documents, or other sources of information, this is generally for the purpose of providing a context for discussing the features of the invention. Unless specifically stated otherwise, reference to such external documents is not to be construed as an admission that such documents, or such sources of information, in any jurisdiction, are prior art, or form part of the common general knowledge in the art.

To those skilled in the art to which the invention relates, many changes in construction and widely differing embodiments and applications of the invention will suggest themselves without departing from the scope of the invention as defined in the appended claims. The disclosures and the descriptions herein are purely illustrative and are not intended to be in any sense limiting.

Although the present invention is broadly as defined above, those persons skilled in the art will appreciate that the invention is not limited thereto and that the invention also includes embodiments of which the following description gives examples.

### BRIEF DESCRIPTION OF THE FIGURES

The present invention will be described with reference to the accompanying figures, in which:

FIG. 1 shows the shelf-life stability of Micrococcus luteus Q24 freeze-dried raw ingredient under refrigerated storage condition of 4° C. P1: Trehalose as lyoprotectant. P2: Blis Technologies Ltd, trimix blend of Trehalose, Maltodextrin and lactitol as lyoprotectant. P3: No lyoprotectant.

FIG. 2 shows the percentage of participants showing a change in skin parameters after use of a composition of the invention compared to baseline using the data derived from a Skin analyser device (Dermo Prime (dp)/viso, CHOWIS, South Korea).

FIG. 3 shows images captured from a participant using a composition of the invention from baseline to day 25. Parameters include pores, spots, impurities, and wrinkles, and parameters were measured using the Skin analyser device (dP/viso, CHOWIS, South Korea)

FIG. 4 shows an example of changes in a range of parameters in a participant before and after application of a composition of the invention over 25 days.

FIG. 5 shows the stability of Q24 in formulations 14, 17, 19, 21, 28, 30, and 32 (see table 1) and in Cetomacrogol cream at 25° C./60% RH.

FIG. 6 shows a scatter plot showing the viabilities (relative to the PBS negative control treatment) of the individual tissues given each dose level of Blis Q24. Treatment groups 5-9 were given Blis Q24 at doses of 105-109 cfu/mL, respectively. The thick horizontal bars show group means and the error bars indicate 95% confidence intervals *, significant difference with P<0.05.

FIG. 7 shows a scatter plot of individual IL-6, IL 8 and IL -18 levels measured in the conditioned media of the tissues on days 0 and 4. Thick horizontal bars are group means and the error bars show the 95% confidence intervals. **, significantly different with P<0.01.

FIG. 8 shows histology of EpiDerm treated for 5 days with Blis Q24 (and PBS and vitamin C—data not shown). Sections were stained with hematoxylin and eosin stain (H&E) and were imaged at 40× magnification. Arrow “B” points to the permeable membrane on which tissues were grown. Tissue layers shown are “C” basal layer, “D” granular layer, “E” stratum corneum. Arrows labelled “A” show nuclear remnants caught in stratum corneum (presence indicates possible acceleration of stratum corneum production).

FIG. 9 shows a scatter plot of individual changes in IL-8 levels measured on days 4 and 5 in the conditioned media of the tissues given combinations of main treatments and ±0.5% SDS. Thick horizontal bars are group means and the error bars show the 95% confidence intervals. ***, significantly different with P<0.001.

FIG. 10 shows skin quality parameters of Blis Q24 “active” composition versus Placebo compared to baseline.

FIG. 11 shows the skin quality parameters of Blis Q24 “Live” composition versus Blis Q24 “Dead” composition compared to baseline.

FIG. 12 shows growth curves of Q24 with potential prebiotics.

FIG. 13 shows growth curves of combinations of prebiotics - olive squalene and pomegranate seed oil; olive squalene and oatmeal flour (colloidal oatmeal); and olive squalene and vitamin E.

FIG. 14 shows the growth of commensal species in presence of prebiotics.

### DETAILED DESCRIPTION OF THE INVENTION

### Definitions

The term “comprising” as used in this specification and claims means “consisting at least in part of”. When interpreting each statement in this specification and claims that includes the term “comprising”, features other than that or those prefaced by the term may also be present. Related terms such as “comprise”, “comprised” and “comprises” are to be interpreted in the same manner.

As used herein the term “and/or” means “and” or “or”, or both.

As used herein “(s)” following a noun means the plural and/or singular forms of the noun.

The general chemical and biological terms used, for example, in the formulae herein have their usual meanings.

The term “subject” as used herein refers to a mammal, including humans, dogs, cats, horses, sheep, cows and other domestic and farm animals.

The unit “cfu/g” means colony-forming units per gram. A colony-forming unit (CFU) is a unit used to estimate the number of viable bacteria in a sample. Determining colony-forming units requires culturing the microbes and counts only viable cells, i.e., those able to multiply and form a visible colony.

The unit “% w/w” means the percentage weight based on the total weight of the composition.

The term “lyoprotectant-free” or “cryoprotectant-free” as used herein means M. luteus has been produced in the absence of a lyoprotectant or cryoprotectant, or both.

The term “improving the appearance of skin or at least one sign of aging in a subject” as used herein means an improvement in at least one parameter commonly used for skin analysis including skin radiance, skin health, skin hydration, pore size, skin softness, skin clarity, moisture levels, sebum levels, appearance of wrinkles, dryness, roughness, dullness, appearance of spots including age spots, and impurities.

### Topical Composition and Methods

Described herein is a method to improve appearance of skin or at least one sign of aging comprising applying to the skin a topical composition comprising Micrococcus luteus Q24. Also described herein is a topical composition comprising Micrococcus luteus Q24 for improving the appearance of skin or at least one sign of aging. Further described herein is use of Micrococcus luteus Q24 in the manufacture of medicament for improving the appearance of skin or at least one sign of aging.

Described herein is a topical composition comprising Micrococcus luteus Q24, a viscosity modifier, a dispersing agent and an oil vehicle, wherein the composition comprises Micrococcus luteus Q24 in an amount of about 1×10⁴ to about 1×10¹⁰ cfu/g.

Also described herein is a topical composition comprising Micrococcus luteus Q24, hydrophobic silica, polysorbate 80, and an oil vehicle.

Also described herein is a topical composition comprising
about 1×10³ to about 1×10² cfu/g Micrococcus luteus Q24,
about 2 to about 10% w/w hydrophobic silica,
about 0.5 to about 2% w/w polysorbate 80, and
a quantity sufficient amount of oil vehicle.

In various embodiments, the oil vehicle is a medium chain triglyceride.

The topical composition described herein may be used for improving the appearance of skin or at least one sign of aging.

Described herein is the use of Micrococcus luteus Q24 in the manufacture of medicament for improving the appearance of skin or at least one sign of aging.

Q24 useful in the present invention has been found by a tissue culture model to be well tolerated, to not elicit an anti-inflammatory response, to have an anti-inflammatory effect, to accelerate the growth of stratum corneum which may help in rejuvenating and hydrating the skin, and reducing pores and wrinkles in skin (see example 9).

### Micrococcus luteus Q24

Micrococcus luteus is a normal bacterial member (commensal) on human skin and is a key bacterium in keeping the balance among the various microbial flora of the skin.

M. luteus Q24 was deposited with Deutsche Sammlung von Mikro organisms Und Zellkulturen GmbH, Braunschweig, Germany, on 10 Mar. 2005, and assigned accession number DSM 17172. M. luteus strain Q24 is described in WO2006104403, incorporated herein by reference.

In various embodiments, the M. luteus is a live probiotic.

In various embodiments, the composition of the invention, which is useful in the method of the invention, comprises Micrococcus luteus Q24 in an amount of about 1×10³ to about 1×10¹² cfu/g. In various embodiments, the composition comprises Micrococcus luteus Q24 in an amount of about 1×10⁴ to about 1×10¹², about 1×10⁵ to about 1×10¹², about 1×10⁶ to about 1×10¹², about 1×10⁷ to about 1×10¹², about 1×10⁸ to about 1×10¹², about 1×10⁴ to about 1×10¹⁰, about 1×10⁵ to about 1×10¹⁰, about 1×10⁶ to about 1×10¹⁰, about 1×10⁷ to about 1×10¹⁰, about 1×10⁸ to about 1×10¹⁰, about 1×10⁴ to about 1×10⁹, about 1×10⁵ to about 1×10⁹, about 1×10⁶ to about 1×10⁹, about 1×10⁷ to about 1×10⁹ cfu/g. In various embodiments, the composition comprises Micrococcus luteus Q24 in an amount of about 1×10⁹ cfu/g.

In various embodiments, Micrococcus luteus Q24 is freeze-dried or lyophilized. In various embodiments, Micrococcus luteus Q24 is provided in a lyoprotectant or cryoprotectant. Lyoprotectants and cryoprotectants are commonly used in the manufacture of products containing probiotics to protect and maintain cell viability. The terms “lyoprotectant” and “cryoprotectant” refer to compositions that protect active ingredients, in this case, Micrococcus luteus Q24. Lyoprotectants protect during drying, while cryoprotectants protect during freezing. The same composition can have both functions, and unless otherwise specified, the terms are used interchangeably herein.

Suitable lyoprotectants or cryoprotectants will be known to a person skilled in the art. In various embodiments, the lyoprotectant is trehalose, or a trimix comprising trehalose, maltodextrin, and lactitol.

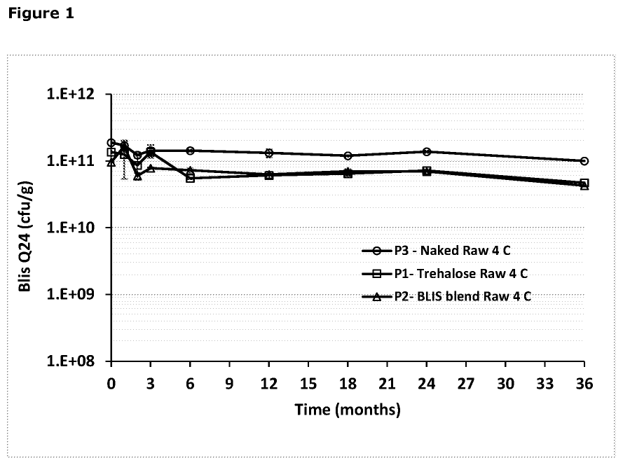

Surprisingly, the applicants have identified that Micrococcus luteus Q24 can also be freeze-dried in the absence of lyoprotectants or cryoprotectants with no significant loss in cell viability as shown in FIG. 1. This result is counter-intuitive, where microorganisms routinely need to be protected during the freeze-drying process. In various embodiments, wherein Micrococcus luteus Q24 is lyoprotectant-free in the composition.

As will be appreciated, this finding leads to significant manufacturing advantages where the freeze-drying process is simplified, and costs are reduced where no lyoprotectants or cryoprotectants need to be used.

### Viscosity Modifier

In various embodiments, the composition comprises a viscosity modifier. Advantageously, the viscosity modifier may be used to modulate the release profile of the probiotic and also modify viscosity of the composition.

In various embodiments, the composition comprises the viscosity modifier in an amount of about 3 to about 15% w/w. For example, the composition may comprise the viscosity modifier in an amount of about 3 to about 10%, or about 3 to about 9%, or about 3 to about 8%, or about 4 to about 15%, or about 4 to about 10%, or about 4 to about 9%, or about 4 to about 8%, or about 5 to about 15%, or about 5 to about 10%, or about 5 to about 9%, or about 5 to about 8% w/w. For example, the composition may comprise the viscosity modifier in an amount of about 3%, about 4%, about 5%, about 6%, about 7%, about 8%, about 9%, about 10%, about 11%, about 12%, about 13%, about 14% or about 15% w/w based on the total weight of the composition. In various embodiments, the composition comprises the viscosity modifier (e.g. hydrophobic silica) in an amount of about 7% w/w.

Suitable viscosity modifiers include, but are not limited to, hydrophobic silica, hydrophilic silica, white beeswax, yellow beeswax, paraffin wax, jojoba wax, microcrystalline wax, ethyl cellulose, stearic acid, xanthan gum, tapioca starch, Carbopol polymer (e.g. 971p, 974p), cocoa butter, shea butter, and a combination of any two or more thereof. Preferably, the viscosity modifier is hydrophobic silica (e.g. Aerosil R972®).

### Dispersing Agent

In various embodiments, the composition comprises a dispersing agent. Advantageously, the dispersing agent may facilitate dispersion of the solid particles in the composition. For example, the dispersing agent may facilitate dispersion of the probiotic in the composition. Additionally, the dispersing agent may assist in emulsification of the oil phase with aqueous phase when mixed together before application. The dispersing agent may be a non-ionic dispersing agent or an amphoteric dispersing agent. Examples of non-ionic dispersing agents include, but are not limited to polysorbate 80 (Tween 80), polysorbate 20 (Tween 20), sorbitan oleate (Span 80), polyoxyl 35 castor oil (Cremaphor EL). Examples of amphoteric dispersing agents include, but are not limited to, lecithin, such as egg lecithin and soybean lecithin.

In various embodiments, the composition comprises the dispersing agent in an amount of about 0.1 to about 5% w/w. For example, the composition may comprise the dispersing agent in an amount of about 0.5 to about 4%, about 0.5 to about 3%, about 0.5 to about 2.5% w/w, about 0.5 to about 2% w/w or about 1 to about 2% w/w. In various embodiments, the composition comprises the dispersing agent (e.g. Tween 80) in an amount of about 1% w/w, or about 2% w/w.

### Oil Vehicle

In various embodiments, the composition comprises an oil vehicle. Suitable oil vehicles include, but are not limited to, medium chain triglycerides and plant oils. Preferably, the medium chain triglyceride is a caprylic/capric triglyceride, such as Miglyol 812N (triglyceride ester of saturated coconut/palm-kernel oil derived caprylic and capric fatty acids and plant derived glycerol). In various embodiments, the plant oil is selected from the group consisting of sunflower oil, canola oil, soybean oil, olive oil, jojoba oil, argan oil, rosehip oil, marula oil, chamomile oil, tamanu oil, grapeseed oil, and a combination of any two or more thereof.

In various embodiments, the composition comprises the non-aqueous carrier in a quantity sufficient (q.s.) amount, i.e. an amount to bring the total % w/w of the composition to 100%. In various embodiments, the composition comprises the non-aqueous carrier in an amount of about 55 to about 95% w/w. For example, the composition may comprise from about 60 to about 95%, or about 60 to about 90%, or about 65 to about 90%, or about 65 to about 90%, or about 70 to about 95%, or about 75 to about 95%, or about 75 to about 90%, or about 80 to about 90%, or about 80 to about 90% w/w, or about 85 to about 95%, or about 85 to about 90%, or about 88 to about 93% w/ w. In various embodiments, the composition comprises the non-aqueous carrier in an amount of 88, 89, 90, 91, 92, or 93% w/w.

In various embodiments, the composition is non-aqueous. In various embodiments, the composition is substantially anhydrous. In various embodiments, the composition comprises less than 7% water, less than 5% water, less than 3% water, less than 2% water, less than 1% water, less than 0.5% water, less than 0.1% water, or less than 0.01% water. In the present composition, water includes absorbed moisture from the environment.

### Additional Probiotics

In various embodiments, the composition further comprises one or more additional probiotics. Suitable additional probiotics include, but are not limited to, Lactobacillus spp. (e.g. L. acidophilus), Limosilactobacilius spp. (e.g. L. reuteri, previously Lactobacillus reuteri), Lacticaseibacillus spp. (e.g. L. rhamnosus, previously Lactobacillus rhamnosus), Ligilactobacillus spp. (e.g. L. salivarius, previously Lactobacillus salivarius), Lactiplantibacillus spp. (e.g. L. plantarum, previously Lactobacillus plantarum), Bifidobacterium spp. (e.g. B. bifidum, B. longum, or B. lactis BB12), Streptococcus spp. (e.g. S. oralis, S. oralis 89a, S. uberis, S. salivarius 24SMB, S. salivarius M18, S. salivarius K12, or S. salivarius DB-B5), and Saccharomyces spp. (e.g. S. boulardii or S. cerevisiae).

S. salivarius K12 was deposited with Deutsche Sammlung von Mikro organismen Und Zellkulturen GmbH, Mascheroder Weg 1 b, D-38124, Braunschweig, Germany on 8 Oct. 1999, and assigned Accession Nos. DSM 13084. S. salivarius M18 was deposited at Deutsche Sammlung von Mikro organismen Und Zellkulturen GmbH, Mascheroder Weg 1 b, D-38124, Braunschweig, Germany on 12 Dec. 2001, and assigned Accession No. DSM 14685.

In various embodiments, the Streptococcus spp. is selected from the group consisting of Streptococcus salivarius K12, Streptococcus salivarius M18, Streptococcus oralis (e.g. S. oralis 89a), Streptococcus salivarius 24SN1B, and a combination of any two or more thereof. In various embodiments, the Streptococcus spp. is selected from the group consisting of Streptococcus salivarius K12, Streptococcus salivarius M18, Streptococcus salivarius 24 SMB, Streptococcus oralis (e.g. S. oralis 89a), Streptococcus salivarius DB-B5, and a combination of any two or more thereof.

In various embodiments, the composition comprises each additional probiotic in an amount of about 1×10³ to about 1×10¹² cfu/g. For example, the composition comprises each additional probiotic in an amount of about 1×10⁴ to about 1×10¹², about 1×10⁵ to about 1×10¹², about 1×10⁶ to about 1×10¹², about 1×10⁷ to about 1×10¹², about 1×10⁸ to about 1×10¹², about 1×10⁴ to about 1×10¹⁰, about 1×10⁵ to about 1×10¹⁰, about 1×10⁶ to about 1×10¹⁰, about 1×10⁷ to about 1×10¹⁰, about 1×10⁸ to about 1×10¹⁰, about 1×10⁴ to about 1×10⁹, about 1×10⁵ to about 1×10⁹, about 1×10⁶ to about 1×10⁹, about 1×10⁷ to about 1×10⁹ cfu/g. In various embodiments, the composition comprises each additional probiotic in an amount of about 1×10⁹ cfu/g.

### Additional Additives

Those persons skilled in the art will appreciate the topical composition may comprise other additives conventionally used in a topical composition, such as a moisturiser. Art skilled readers will further appreciate that additives need to be compatible with probiotic viability and efficacy. Such additives may provide or improve a therapeutic, cosmetic, stability, and/or appearance property of the composition. Examples of suitable additives include, but are not limited to, an inhibitory activity enhancer, a buffering agent, an antibacterial agent, a prebiotic, a fragrance, an antioxidant, a colourant, a skin protective agent, an antimicrobial, an aluminium salt, a mineral pigment, an odour absorbant or neutraliser, or a sunscreen agent. Such additives may be included in the composition of the invention in amounts typical for topical formulations. A variety of pharmaceutically acceptable additives suitable for topical application of viable or lyophilized bacteria are well known in the art. A skilled worker will appreciate that any additional additive should not be inhibitory towards Micrococcus luteus Q24.

A skilled worker will also appreciate that some additives may have dual function, for example, an antioxidant or skin protective agent may also function as a prebiotic.

In various embodiments, the composition further comprises an inhibitory activity enhancer, a buffering agent, an antibacterial agent, a prebiotic, a fragrance, an antioxidant, a colourant, a skin protective agent, an antimicrobial, an aluminium salt, a mineral pigment, an odour absorbant or neutraliser, a sunscreen agent, and a combination of any two or more thereof.

In various embodiments, the inhibitory activity enhancer is selected from the group consisting of sodium chloride, ethylenediaminetetraacetic acid, arginine, calcium carbonate, and a combination of any two or more thereof.

In various embodiments, the buffering agent is selected from the group consisting of calcium carbonate, magnesium carbonate, sodium hydrogen phosphate, sodium dihydrogen phosphate, dipotassium hydrogen phosphate, potassium dihydrogen phosphate, magnesium carbonate, urea, hydrated aluminium oxide, bentonite clay, kaolin clay, and a combination thereof.

In various embodiments, the antibacterial agent is selected from the group consisting of xylitol, erythritol, epidermin, nisin, salivaricin A, salivaricin Al, salivaricin A2, salivaricin B, and a combination thereof. In various embodiments, the antibacterial agent is selected from the group consisting of xylitol, erythritol, epidermin, nisin, salivaricin A, salivaricin Al, salivaricin A2, salivaricin B, salivaricin 9, salivaricin MPS, and a combination thereof.

In various embodiments, the composition comprises a prebiotic. Prebiotics may enhance the growth of Q24, enhance antimicrobial activity, or enhance the production of compounds to enhance skin quality parameters.

In various embodiments, the prebiotic is selected from the group consisting of Manuka honey, olive squalene, pomegranate seed oil, flax seed oil, coconut oil, colloidal oatmeal, vitamin E, vitamin C, olive oil, hyaluronic acid, calendula oil, almond oil, tomato oil, allantoin, aloe vera powder, colloidal oatmeal, xylitol, yeast extract, fructooligosaccharide powder, fructooligosaccharide liquid, fructooligosaccharides, galactooligosaccharides, xylooligosaccharides, and a combination of any two or more thereof.

In various embodiments, the prebiotic is selected from the group consisting of yeast extract, cysteine, maltodextrin, inulin, liquorice root, liquorice extract, honey, and a combination of any two or more thereof. In various embodiments, the prebiotic is selected from the group consisting of yeast extract, cysteine, maltodextrin, inulin, liquorice root, liquorice extract, and a combination of any two or more thereof.

In various embodiments, the prebiotic may be an oil prebiotic or a powder prebiotic. In various embodiments, the composition comprises an oil prebiotic and/or a powder prebiotic. In various embodiments, the oil prebiotic is selected from the group consisting of olive squalene, pomegranate seed oil, flax seed oil, coconut oil, vitamin E, olive oil, calendula oil, almond oil, tomato oil, fructooligosaccharide liquid, and a combination of any two or more thereof. In various embodiments, the powder prebiotic is selected from the group consisting of Manuka honey, colloidal oatmeal, vitamin C, hyaluronic acid, allantoin, aloe vera powder, colloidal oatmeal, xylitol, yeast extract, fructooligosaccharide powder, fructooligosaccharides, galactooligosaccharides, xylooligosaccharides, and a combination of any two or more thereof.

In various embodiments, the prebiotic is a combination of olive squalene and pomegranate seed oil; olive squalene and vitamin E; or olive squalene, pomegranate seed oil and vitamin E. The inventors have surprisingly identified that combinations of olive squalene and pomegranate seed oil or olive squalene and vitamin E have a synergistic effect on the growth of Micrococcus luteus Q24.

In various embodiments, the composition comprises about 0.1% to about 10% w/w prebiotic, for example from about 0.1% to about 8%, or about 0.1% to about 6%, or about 0.1% to about 5%, or about 0.3% to about 8%, or about 0.3% to about 6%, or about 0.3% to about 5%, or about 0.5% to about 8%, or about 0.5% to about 6%, or about 0.5% to about 5%, or about 1% to about 8%, or about 1% to about 6%, or about 1% to about 5% w/w prebiotic.

In various embodiments, the composition comprises about 0.1% to about 35% w/w prebiotic, for example from about 0.1% to about 30%, about 0.1% to about 25%, or about 0.1% to about 20%, or about 0.1% to about 18%, or about 0.1% to about 16%, or about 0.1% to about 15%, or about 0.1% to about 12%, or about 0.1% to about 10%, or about 0.1% to about 8%, or about 0.1% to about 6%, or about 0.1% to about 5%, or about 0.1% to about 2%, or about 0.3% to about 35%, or about 0.3% to about 30%, or about 0.3% to about 25%, or about 0.3% to about 20%, or about 0.3% to about 18%, or about 0.3% to about 16%, or about 0.3% to about 15%, or about 0.3% to about 12%, or about 0.3% to about 10%, or about 0.3% to about 8%, or about 0.3% to about 6%, or about 0.3% to about 5%, or about 0.3% to about 2%, or about 0.5% to about 35%, or about 0.5% to about 30%, or about 0.5% to about 25%, or about 0.5% to about 20%, or about 0.5% to about 18%, or about 0.5% to about 16%, or about 0.5% to about 15%, or about 0.5% to about 12%, or about 0.5% to about 10%, or about 0.5% to about 8%, or about 0.5% to about 6%, or about 0.5% to about 5%, or about 0.5% to about 2%, or about 0.5% to about 35%, or about 1% to about 30%, or about 1% to about 25%, or about 1% to about 20%, or about 1% to about 18%, or about 1% to about 16%, or about 1% to about 15%, or about 1% to about 12%, or about 1% to about 10%, or about 1% to about 8% , or about 1% to about 6%, or about 1% to about 5%, or about 0.5% to about 35%, or about 3% to about 30%, or about 3% to about 25%, or about 3% to about 20%, or about 3% to about 18%, or about 3% to about 16%, or about 3% to about 15%, or about 3% to about 12%, or about 3% to about 10%, or about 3% to about 8%, or about 3% about 6%, or about 3% to about 5%, or about 4% to about 30%, or about 4% to about 25%, or about 4% to about 20%, or about 4% to about 18%, or about 4% to about 16%, or about 4% to about 15%, or about 4% to about 12%, or about 4% to about 10%, or about 4% to about 8%, or about 4% to about 6%, or about 4% to about 5% w/w prebiotic.

In various embodiments, the composition comprises an oil prebiotic in an amount of about 35% w/w prebiotic, for example from about 0.1% to about 30%, about 0.1% to about 25%, or about 0.1% to about 20%, or about 0.1% to about 18%, or about 0.1% to about 16%, or about 0.1% to about 15%, or about 0.1% to about 12%, or about 0.1% to about 10%, or about 0.1% to about 8%, or about 0.1% to about 6%, or about 0.1% to about 5%, or about 0.1% to about 2%, or about 0.3% to about 35%, or about 0.3% to about 30%, or about 0.3% to about 25%, or about 0.3% to about 20%, or about 0.3% to about 18%, or about 0.3% to about 16%, or about 0.3% to about 15%, or about 0.3% to about 12%, or about 0.3% to about 10%, or about 0.3% to about 8%, or about 0.3% to about 6%, or about 0.3% to about 5%, or about 0.3% to about 2%, or about 0.5% to about 35%, or about 0.5% to about 30%, or about 0.5% to about 25%, or about 0.5% to about 20%, or about 0.5% to about 18%, or about 0.5% to about 16%, or about 0.5% to about 15%, or about 0.5% to about 12%, or about 0.5% to about 10%, or about 0.5% to about 8%, or about 0.5% to about 6%, or about 0.5% to about 5%, or about 0.5% to about 2%, or about 0.5% to about 35%, or about 1% to about 30%, or about 1% to about 25%, or about 1% to about 20%, or about 1% to about 18%, or about 1% to about 16%, or about 1% to about 15%, or about 1% to about 12%, or about 1% to about 10%, or about 1% to about 8%, or about 1% to about 6%, or about 1% to about 5%, or about 0.5% to about 35%, or about 3% to about 30%, or about 3% to about 25%, or about 3% to about 20%, or about 3% to about 18%, or about 3% to about 16%, or about 3% to about 15%, or about 3% to about 12%, or about 3% to about 10%, or about 3% to about 8%, or about 3% to about 6%, or about 3% to about 5%, or about 4% to about 30%, or about 4% to about 25%, or about 4% to about 20%, or about 4% to about 18%, or about 4% to about 16%, or about 4% to about 15%, or about 4% to about 12%, or about 4% to about 10%, or about 4% to about 8%, or about 4% to about 6%, or about 4% to about 5% oil prebiotic.

In various embodiments, the composition comprises a powder prebiotic in an amount of about 0.1% to about 20%, or about 0.1% to about 18%, or about 0.1% to about 16%, or about 0.1% to about 15%, or about 0.1% to about 12%, or about 0.1% to about 10%, or about 0.1% to about 8%, or about 0.1% to about 6%, or about 0.1% to about 5%, or about 0.1% to about 2%, or about 0.3% to about 20%, or about 0.3% to about 18%, or about 0.3% to about 16%, or about 0.3% to about 15%, or about 0.3% to about 12%, or about 0.3% to about 10%, or about 0.3% to about 8%, or about 0.3% to about 6%, or about 0.3% to about 5%, or about 0.3% to about 2%, or about 0.5% to about 20%, or about 0.5% to about 18%, or about 0.5% to about 16%, or about 0.5% to about 15%, or about 0.5% to about 12%, or about 0.5% to about 10%, or about 0.5% to about 8%, or about 0.5% to about 6%, or about 0.5% to about 5%, or about 0.5% to about 2%, or about 1% to about 20%, or about 1% to about 18%, or about 1% to about 16%, or about 1% to about 15%, or about 1% to about 12%, or about 1% to about 10%, or about 1% to about 8%, or about 1% to about 6%, or about 1% to about 5%, or about 3% to about 20%, or about 3% to about 18%, or about 3% to about 16%, or about 3% to about 15%, or about 3% to about 12%, or about 3% to about 10%, or about 3% to about 8%, or about 3% to about 6%, or about 3% to about 5%, or about 4% to about 20%, or about 4% to about 18%, or about 4% to about 16%, or about 4% to about 15%, or about 4% to about 12%, or about 4% to about 10%, or about 4% to about 8%, or about 4% to about 6%, or about 4% to about 5% w/w powder prebiotic.

In a particular embodiment, the composition comprises from about 0.1 to about 30% w/w, or from about 3% to about 8% w/w, or from about 4% to about 6% w/w, or about 5% w/w olive squalene. In a particular embodiment, the composition comprises from about 0.1 to about 30% w/w, or from about 3% to about 8% w/w, or from about 4% to about 6% w/w, or about 5% w/w olive squalene. In a particular embodiment, the composition comprises about 5% w/w flax seed oil. In a particular embodiment, the composition comprises about 1% w/w colloidal oatmeal. In a particular embodiment, the composition comprises from about 0.1 to about 2% w/w, or about 0.2 to about 1.5%, or about 0.2 to about 0.6%, or about 0.3%, or about 0.5 to about 1.3%, or about 0.8 to 1.2%, or about 1% w/w vitamin E.

The inventors have unexpectedly identified that combination of the prebiotics olive squalene and pomegranate seed oil; and the combination of the prebiotics olive squalene and vitamin E are synergistic. Accordingly, in a particular embodiment, the composition comprises from about 0.1% to about 10% w/w olive squalene, about 3% to about 8% w/w, or about 4 to about 7% w/w, or about 5% w/w, or about 0.5% to about 3% w/w, or about 0.5 to about 2% w/w, or about 1% w/w olive squalene, and from about 0.1 to about 10% w/w pomegranate seed oil, or about 2 to about 8% w/w, or about 3 to about 7% w/w, or about 4 to about 6% w/w, or about 3 to about 5% w/w, or about 5% w/w, or about 0.5% to about 3% w/w, or about 0.5 to about 2% w/w, or about 1 % w/w pomegranate seed oil. In a particular embodiment, the composition comprises from about 0.1% to about 10% w/w olive squalene, about 3% to about 8% w/w olive squalene, or about 4 to about 7% w/w olive squalene, for example from about 5% w/w olive squalene, and from about 0.1 to about 3% w/w, or about 0.1 to about 2% w/w, or about 0.2 to about 1.5%, or about 0.2 to about 0.6%, or about 0.3%, or about 0.5 to about 1.3%, or about 0.8 to 1.2%, or about 1% w/w vitamin E.

In a particular embodiment, the composition comprises from about 0.1% to about 10% w/w olive squalene, or about 3% to about 8% w/ w, or about 4 to about 7% w/w, or about 5% w/w, or about 0.5% to about 3% w/w, or about 0.5 to about 2% w/w, or about 1% w/w olive squalene; and from about 0.1 to about 10% w/w pomegranate seed oil, or about 2 to about 8% w/w, or about 3 to about 7% w/w, or about 4 to about 6% w/w, or about 3 to about 5% w/w, or about 5% w/w, or about 0.5% to about 3% w/w, or about 0.5 to about 2% w/w, or about 1% w/w pomegranate seed oil; and from about 0.1 to about 3% w/w, or about 0.1 to about 2% w/w, or about 0.2 to about 1.5%, or about 0.2 to about 0.6%, or about 0.3%, or about 0.5 to about 1.3%, or about 0.8 to 1.2%, or about 1% w/w vitamin E.

In various embodiments, the fragrance is selected from the group consisting of rose water, orange blossom, rose gardenia, peony, white jasmine, ylang ylang oil, geranium oil, rose oil, and a combination of any two or more thereof. The fragrance may be added as an oil or a powder.

In various embodiments, the antioxidant is selected from the group consisting of vitamin E, resveratrol, squalene, vitamin C, β-carotene, retinyl acetate, retinyl palmitate, retinol, niacinamide, green tea, green tea extract, caffeine, and a combination of any two or more thereof. In various embodiments, the antioxidant is selected from the group consisting of vitamin E, resveratrol, squalene (for example, olive squalene), vitamin C, β-carotene, retinyl acetate, retinyl palmitate, retinol, niacinamide, pomegranate seed oil, flaxseed oil, and a combination of any two or more thereof.

In various embodiments, the skin protective agent is selected from the group consisting of ceramide, collagen, elastin, coenzyme Q10, hydrolysed collagen, hyaluronic acid, sodium hyaluronate, retinol, palmitoyl tripeptide-1, palmitoyl tetrapeptide-7, palmitoyl tetrapeptide-38, acetyl hexapeptide-8, heptapeptide-14, heptapeptide-15-palmitate, palmitoyl tetrapeptide-7, palmitoyl tripeptide-1, alpha-hydroxy acids (e.g. lactic acid, glycolic acid), beta-hydroxy acids (e.g. salicylic acid), vitamin B5, seaweed, seaweed extract, silicone, xylitol, oat extract, oatmeal powder, colloidal oatmeal, aloe vera, and a combination of any two or more thereof. In various embodiments, the skin protective agent is selected from the group consisting of ceramide, collagen, elastin, coenzyme Q10, hydrolysed collagen, hyaluronic acid, sodium hyaluronate, retinol, palmitoyl tripeptide-1, palmitoyl tetrapeptide-7, palmitoyl tetrapeptide-38, acetyl hexapeptide-8, heptapeptide-14, heptapeptide-15-palmitate, palmitoyl tetrapeptide-7, palmitoyl tripeptide-1, alpha-hydroxy acids (e.g. lactic acid, glycolic acid), beta-hydroxy acids (e.g. salicylic acid), vitamin B5, seaweed, seaweed extract, silicone, xylitol, oat extract, oatmeal powder, colloidal oatmeal, aloe vera, sunscreen, and a combination of any two or more thereof.

In various embodiments, the antimicrobial is selected from the group consisting of zinc, salicylic acid, azelaic acid, benzoyl peroxide, and a combination thereof. In various embodiments, the antimicrobial is selected from the group consisting of zinc, azelaic acid, benzoyl peroxide, and a combination thereof.

In various embodiments, the aluminium salt is aluminium chlorohydrate salt, or other salts commonly used in antiperspirants.

In various embodiments, the mineral pigment is zinc oxide and/or titanium dioxide. Mineral pigments are known to protect against UV radiation e.g. from the sun.

In various embodiments, the odour absorbant or neutraliser is zinc ricinoleate or sodium lauryl sarcosinate.

In various embodiments, the sunscreen agent is oxybenzone, avobenzone, octisalate, octocrylene, homosalate, or octinoxate. Mineral sunscreens use zinc oxide and/or titanium dioxide.

A skilled worker will also understand that when the composition is applied in combination with an aqueous phase, the additional additives described may also be provided in the aqueous phase.

### Improving Appearance of Skin or Signs of Aging

The compositions described herein are useful for improving the appearance of skin or improving signs of aging of the skin. Improving the appearance of skin or a sign of aging may include effects that include, but are not limited to, that skin looks more radiant, skin looks healthier, skin feels more hydrated, pores are reduced in size, skin feels softer, skin looks clearer, moisture is increased, sebum production is decreased, and reducing the appearance of wrinkles, dryness, roughness, dullness, spots including age spots, and impurities.

Pore size, skin moisture content, sebum productions, wrinkles, spots, and impurities may all be measured using a Skin analyser device (eg. (Dermo Prime (dp)/viso, CHOWIS, South Korea). An AI powered skin analyser device quantitatively measures changes in various skin parameters such as hydration, sebum, pores, wrinkles, spots/pigmentation, impurities, keratin, skin tone, blackheads, and skin sensitivity. The device is equipped with a moisture sensor and utilises advanced optic technology with interchangeable lenses to measure up to 10 different skin measurements and performs accurate analysis of high resolution images via the DermoBella app installed in android or OS tablets or smartphones. The device is used according to the protocols as set out in the Examples herein. Skin parameter measurements are taken at the same skin site each time.

The degree of impurities such as redness can be analysed by measuring the amount of porphyrin. It is indicated as scarlet, orange light in response to a specific wavelength range of UV light.

Impurities are seen as either a scarlet colour or a yellow-green colour. These can be analysed separately, but the device combines the two and classifies them as impurities. The index is computed by a percentage against image size. There are no arbitrary values that classify the degree of redness severity based on the amount of porphyrin detected.

### Form of the Topical Composition

In various embodiments, the composition has a viscosity of from about 20,000 to about 500,000 cp at 25° C., for example from about 30,000 to about 100,000 cp at 25° C. In various embodiments, the composition has a viscosity of from about 20,000 to about 2,000,000 cp at 25° C.

Viscosity may be measured at 25° C. using a Brookfield LVDVI Prime using Brookfield Helipath Spindle (S94 or S95 or S96) set at 0.5 RPM. A skilled worker will appreciate other methods that can be used to measure viscosity and that viscosity measurements may vary depending on the method used.

In various embodiments, the composition has a shelf-life of at least 6 months at 25° C. at 60% RH (relative humidity). In various embodiments, the composition has a shelf-life of at least 12 months at 25° C. at 60% RH (relative humidity).

### Two-Phase Composition

Aqueous compositions generally require the addition of preservatives to prevent growth of pathogens. These preservatives are typically non-selective and lead to probiotic cell death, and therefore loss in efficacy within a short time. Additionally, Micrococcus luteus Q24 is not as stable in aqueous formulations. However, it may be advantageous to apply the topical composition of the invention with an aqueous composition to, for example, improve the spreadability of the composition, improve the sensory features of the composition (e.g. feels less oily), and/or act as a vehicle without affecting the stability or viability of Micrococcus luteus Q24.

In various embodiments, when applied, the composition is applied in combination with an aqueous phase.

Suitable aqueous phases will be apparent to a skilled worker. The aqueous phase may comprise a suitable combination of solvents, emollients, humectants, emulsifying agents, preservatives, viscosity modifiers, prebiotics, and fragrance. Commercially available moisturisers are an example of a suitable aqueous phase.

Components of aqueous phases include, but are not limited to, water, sodium CMC and microcrystalline cellulose (Vivapur MCG 811), preservative (e.g. phenoxyethanol and ethylhexylglycerin), glycerin, sodium surfactin, xantham gum, fragrance, coco-caprylate, cetearyl alcohol, glyceryl stearate, cetearyl wheat straw glycosides, phenoxyethanol, and sodium stearoyl glutamate.

The aqueous phase may also comprise additional additives as outlined above, such as prebiotics or antioxidants.

Also described herein is a two-phase composition comprising an oil phase and an aqueous phase, wherein the oil phase comprises Micrococcus luteus Q24, a viscosity modifier, a dispersing agent and an oil vehicle. Described herein is a two-phase composition comprising a separate oil phase and a separate aqueous phase, wherein the oil phase comprises Micrococcus luteus Q24, a viscosity modifier, a dispersing agent and an oil vehicle. The composition may comprise any of the features previously described. In various embodiments, the oil phase is applied simultaneously with the aqueous phase.

The two-phase composition may be packaged in a dual chamber bottle containing separate chambers or reservoirs for the oil phase and the aqueous phase, with a nozzle designed to dispense the two phases together at the time of application. The phases are stored separately in the chambers and combined upon application. An example of a suitable dispenser is described in U.S. Pat. No. 10,384,224.

### Kit

Described herein is a kit comprising a topical composition comprising Micrococcus luteus Q24 and an aqueous composition.

In various embodiments, the kit comprises a dispensing system having a first reservoir and a second reservoir, wherein the first reservoir comprises an oil phase comprising Micrococcus luteus Q24 and the second reservoir comprises an aqueous phase. A dual chamber bottle such as described in U.S. Pat. No. 10,384,224 is suitable for inclusion in the kit.

The kit may also be provided with instructions for use.

### Method of Preparing Topical Composition

In one aspect, the invention provides a method of manufacturing a topical composition comprising Micrococcus luteus Q24 comprising the steps of:
a) mixing an oil vehicle and dispersing agent,
b) adding Micrococcus luteus Q24 and a viscosity modifier to the mixture from step a),
c) homogenising the mixture from step b) to provide the composition.

The topical composition described herein may be prepared by first mixing an oil vehicle and dispersing agent, adding Micrococcus luteus Q24 to the mixture with constant swirling, adding a viscosity modifier to the mixture, and homogenising the mixture e.g. for about 1 to 3 minutes, to provide the composition. In various embodiments, the Micrococcus luteus Q24 is added before the viscosity modifier. In other embodiments, the viscosity modifier is added before the Micrococcus luteus Q24. In various embodiments, the mixture is homogenized with a high shear homogeniser or an overhead stirrer.

Advantageously, the method does not comprise a heating step. In various embodiments, the method is carried out without heating. Micrococcus luteus Q24 has been found to be heat-sensitive. Heating results in loss of cell viability. The applicants have therefore developed alternative compositions that allow efficient manufacturing of the composition while maintaining optimal viability of the probiotic.

The Micrococcus luteus Q24 may be milled or sieved to a particle size of less than 250 μm prior to adding to the mixture. In various embodiments, the particle size of Micrococcus luteus Q24 is less than about 250 μm, or less than about 100 μm. Suitable milling and sieving techniques will be apparent to a skilled worker. For example, the Micrococcus luteus Q24 may be milled using a dry powder comill (e.g. Quadro Powder Mills, such as U10 or the U21). Smaller particle sizes may be advantageous for dispensing the oil phase from the two-phase dispenser, provide improved spreadability of the composition, enhance dispersibility when combined with the aqueous phase, and provide improved sensory properties of the composition i.e. is not grainy or gritty.

In various embodiments, Micrococcus luteus Q24 have a particle size (Dv90) of less than about 300 μm, or less than about 250 μm. Dv90 may be measured by the laser diffraction method for particle size analysis of dry powder dispersion (Malvern Instruments, USA). A skilled worker will appreciate that particles with a Dv90 of less than 300 μm may entirely pass through a 250 μm sieve.

### Methods of Use

The invention relates to a method of improving the appearance of skin or at least one sign of aging in a subject in need thereof. The method comprises applying to the skin of a subject in need thereof a topical composition of the invention. The compositions of the invention may be used in a two-phase moisturising cream, as an oil phase serum alone, as a facial cream or all over body moisturiser, a spray, or as a spot-on cream. The composition may be applied in an amount appropriate to address the skin issue of the subject. A skilled worker will appreciate the composition may be applied to a subject of any age from the very young to the very old. The amount applied may vary according to the subjects age, sex, and issue being addressed. A typical application regime may comprise application of the composition of the invention monthly, weekly, daily, or between one and four times daily.

A typical dose of the composition when applied to the face or used as a spot on formulation is typically 0.09 to 0.20 grams, and more commonly 0.10 to 0.15 grams. A dose will typically comprise from about 1×10⁵ to about 1×10⁹ cfu. Larger amounts will of course be used for body moisturizing. The composition may be applied by hand, or dispensed from a dual chamber bottle or other device, A skilled worker will understand that the amount dispensed will depend on the concentration of Micrococcus luteus Q24 in the composition to be applied, and the form in which it is applied.

The compositions of the invention may be used in the same way as a conventional moisturiser, and in any daily skin-care regime. A typical regime may comprise use of the composition of the invention once or twice daily, and usually after cleansing. Use of the composition for extended periods, or for time-limited periods e.g. for one to two months is contemplated.

### EXAMPLES

### Materials

Equipment: Ingredients of the oil phase formulation were mixed using the homogenizer (IKA Ultra Turrax T25, John Morris group, New Zealand). For the clinical trial, oil phase was packaged into dual cylinder single dispensing bottles (Neomix, Salient, France).

Chemicals: Blis Q24 was fermented and freeze dried by FoodBowl Auckland. Blis lyoprotectant blend was prepared by Callaghan innovation (June 2017). Several skin pathogen strains were procured from −80° C. freezer located at Blis Technologies Lab. Medium chain triglyceride (Caprylic/Capric Triglyceride) e.g. Miglyol 812 (Ph Eur grade) or Radia 7104 were purchased from Sasol, Hamburg, Germany or Olean, Malaysia respectively. Tween 80 (Ph Eur grade) was purchased from Sigma (New Zealand). Hydrophobic silica (Aerosil R 972 (Pharma grade) was purchased from Evonik, Germany (supplied by Chemiplas, Auckland, New Zealand). Aqueous cream prepared inhouse at Blis Technologies and aqueous moisturiser was supplied by Shieling laboratories New Zealand. Distilled water was used for sample and culture preparations. All other chemicals and solvents were of analytical grade (Sigma, New Zealand). Plastic bottles for storing Q24 oil phase and aqueous phase for clinical trial and stability studies were purchased from Neomix, France. Skin analyser device (dpViso, Dermobella app) was purchased from CHOWIS, North Korea. Samsung Tablet 10.1 was purchased from PB Technologies, New Zealand.

### Example 1: Blis Q24 Raw Ingredient Size Optimization for Inclusion in the Oil Phase

Blis Q24 raw ingredient had visibly large particle size >500 μm. For the formulation of oil phase, the intended particle size was to be less than <250 μm to allow consistent dispensing from the dual cylinder single dispensing bottles (Neomix, France) and sensory aesthetic upon application on the skin. A filter bag (BagPage+Full-page filter bag with Microperforated filter, <250 μm) (Interscience, New Zealand) was used. Blis Q24 was placed inside the inner mesh pocket/sieve (250 μm) of the filter bag and finely sieved material (<250 μm) was collected in the outer pocket following 5 min of hand shaking. If desired, light pressure using a rolling pin was applied to the raw ingredient to further break up any large aggregates. A small cut was then made in the outer pocket of the stomacher bag and the separated sieved (<250 μm) and non-sieved (>250 μm) material was collected and stored separately.

### Example 2: Method of Preparing Topical Composition

In a 250 ml glass beaker, the required amount of vehicle (see Table 1, MCT) was weighed followed by addition of dispersing agent Tween 80. The beaker was then swirled gently by hand until a hazy mixture is formed. Blis Q24 and hydrophobic silica were then added to the MCT/Tween 80 mixture with constant swirling to obtain a homogenous mix followed by dispersion using high shear or over-head homogeniser to disperse all ingredients homogenously.

TABLE 1
Example formulations

| | | | | | | | | | Aqueous | | |
|---|---|---|---|---|---|---|---|---|---|---|---|
| | | | | | | | | | Cream | | |
| | | Blis | | | | Capric/ | | | BP | | |
| | | Q24 | Hydrophobic | | | caprylic | | | (SLS | | |
| | Blis | (Blis | silica | Tween | Sodium | triglyceride | Olive | Coconut | free) | | |
| | Q24 | Blend | (AR972) | 80 | surfactin | (MCT) | oil | oil | Aq. | Water | Glycerol |
| Ingredient | naked | Lyo) | Viscosity | Dispersing | Dispersing | Oil | Oil | Oil | Cream | Aq. | Polar |
| Function | Active | Active | modifier | agent | agent | Vehicle | Vehicle | Vehicle | base | Vehicle | solvent |
| BLT17Q24- | 1% | — | — | — | — | — | — | — | 99% | — | — |
| 1 | | | | | | | | | | | |
| BLT17Q24- | 1% | — | — | — | — | — | — | — | — | 99% | — |
| 2 | | | | | | | | | | | |
| BLT17Q24- | 1% | — | — | — | — | — | — | 99% | — | — | — |
| 5 | | | | | | | | | | | |
| BLT17Q24- | 1% | — | — | — | — | 99% | — | — | — | — | — |
| 7 | | | | | | | | | | | |
| BLT17Q24- | — | 1% | 5% | 1% | — | 93% | — | — | — | — | — |
| 14 | | | | | | | | | | | |
| BLT17Q24- | — | 1% | 10% | 2% | — | 87% | — | — | — | — | — |
| 17 | | | | | | | | | | | |
| BLT17Q24- | — | 1% | 5% | 1% | — | — | 93% | — | — | — | — |
| 19 | | | | | | | | | | | |
| BLT17Q24- | — | 1% | 10% | 2% | — | — | 87% | — | — | — | — |
| 21 | | | | | | | | | | | |
| BLT17Q24- | 2% | — | 6% | 1% | — | 91% | — | — | — | — | — |
| 25 | | | | | | | | | | | |
| BLT17Q24- | — | 2% | 7% | 1% | — | 90% | — | — | — | — | — |
| 28 | | | | | | | | | | | |
| BLT17Q24- | — | 2% | 8% | 1% | — | 89% | — | — | — | — | — |
| 29 | | | | | | | | | | | |
| BLT17Q24- | — | 1% | 5% | 1% | — | 87% | — | — | — | — | 10% |
| 30 | | | | | | | | | | | |
| BLT17Q24- | — | 2.5% | — | — | 0.25% | 87.25% | — | — | — | — | 10% |
| 31 | | | | | | | | | | | |
| BLT17Q24- | 2% | — | 7% | 1% | — | 90% | — | — | — | — | — |
| 32 | | | | | | | | | | | |

### Example 3: Stability Testing

Stability of formulations was tested as per ICH recommended storage conditions of temperature and humidity following guidelines for stability testing (ICH Q1AR2) by storing under refrigerated 5° C.±3° C. and/or real time condition of 25° C.±2° C./60±5% RH in glass bottles or dual chamber bottles.

For formulation BLT17Q24-2 (aqueous cream) a decline was observed with less than 1 log reduction within 2 months when stored at 5° C.±3° C. A dramatic decrease in viable cell count was observed within one week when the formulations were stored under 25° C.±2° C./60±5% RH for formulations BLT17Q24-2 (aqueous cream), BLT17Q24-5 (coconut oil), and BLT17Q24-1 (water). Formulation BLT17Q24 -7 (medium chain triglyceride) was stable for at least 6 months (when the testing concluded).

Formulations BLT17Q24-30 and BLT17Q24-31 containing glycerol were not stable irrespective of the oil type. Formulations BLT17Q24-14 (MCT vehicle), BLT17Q24-17 (MCT vehicle), BLT17Q24-19 (olive oil vehicle), BLT17Q24-21 (olive oil vehicle) were found to be stable for at least 9 months at 25° C.±2° C./60±5% RH in glass bottles (FIG. 5).

BLT17Q24-25 (MCT vehicle) was stable for at least 6 months in a dual chamber bottle at 25° C.±2° C./60±5% RH and at 30° C.±2° C./65 ±5% RH, and for at least 9 months in a glass vial at 5° C.±3° C., and at least 6 months at 25° C.±2° C./60±5% RH and 40° C.±2° C./75±5% RH. It is expected that the formulations would be stable for at least 2 years.

### Example 4: Aqueous Phase

An example aqueous phase is provided.

TABLE 2
Aqueous formulation

| Ingredient | Function | % w/w |
|---|---|---|
| Sterile deionised water | Solvent | 83.5% |
| Sodium CMC + | Viscosity controlling | 4% |
| Microcrystalline Cellulose | | |
| (Vivapur MCG 811) | | |
| Preservative (Phenoxyethanol + | Preservative | 1% |
| Ethylhexylglycerin) - | | |
| Euxylk PE9010 | | |
| Glycerin | Humectant | 10% |
| Sodium Surfactin | Emulsifying agent | 0.5% |
| Xanthan Gum Smooth | Prebiotic | 0.5% |
| Fragrance - Perfume Oil | Aroma | 0.5% |
| Botanic Light Fresh DV4737 | | |
| Total | | 100% |

### Example 5: In Vivo Trials

A cosmetic trial using formulation BLT17Q24-28 was carried out in November-December 2020 involving adult human volunteers (n=10). The participants were all female and were aged between 22 and 60. The trial design involved measurement of skin parameters using a novel skin analyser and swab sample collection for determining colonisation efficacy of Blis Q24 face moisturiser.

### Clinical Trial Sample Collection

Each participant was assessed in the same room each time and followed the same procedure each time point. For the measurement of skin parameters using the skin analyser, the device sensor was placed on the left cheek in line with the corner of the eye and the tip of the nose. This method was kept consistent for each time point so that the readings were taken approximately from the same area on the cheek or forehead as instructed by the device app. Wrinkles were measured by placing the device camera on the corner of the eye. Moisture/hydration status was measured by placing the device on the forehead (T-zone) and then on the cheek (U-zone).

Sebum/oiliness was measured by placing the T-zone side of the sebum paper (labelled by CHOWIS) on the forehead and then placing the U-zone side of the sebum paper on the cheek. The analyser camera was then used to take picture of the sebum on the sebum paper. For the measurement of different parameters, the device sensor was not moved when the images were taken and when possible 3 images were captured for each parameter. Measurements were collected in a step wise manner and follow the order of moisture, sebum, pore size, spots, impurities, wrinkles based on instruction on the device software.

### Trial Results

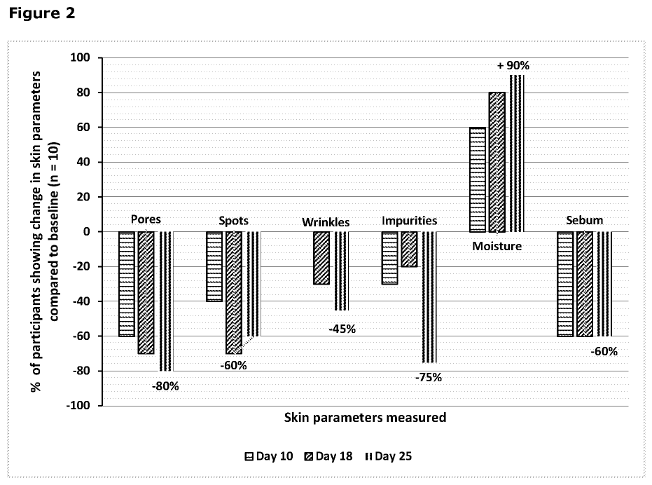

FIG. 2 shows the change in different skin parameters following application on the face of two pumps of oil phase (BLT17Q24-28)+face moisturiser combo using single dispenser bottle for 25 days. Each pump delivered on an average 0.05 g thereby delivering a total dose of this equates to a total dose of=0.200g per day (˜0.05 g×2 pumps twice daily). The total cell count deposited on the face per dose (two pumps 0.1 g) was 3.29E+8 CFU/0.1 g or 6.6E+8 CFU/day for twice daily dose. These are based on the initial oil phase cell count at Day 0=3.29E+9 CFU/g.

Compared to baseline, a consistent reduction in pore size, spots, wrinkles (in the corner of eye), impurities (porphyrins) and sebum levels and a consistent increase in moisture levels (T-zone) was observed at each time point (10, 18, 25 days) in 60%- 90% of participants suggesting the efficacy of Blis Q24 hydrating serum face moisturiser.

FIG. 2 describes the percentage change in skin parameters in all participants from baseline to Day 25. Moisture/hydration levels had the largest percentage change within the study participants with an overall increase of 90%. Wrinkles had an overall decrease of 45%. Sebum production was reduced by 60% after 10 days. Changes in sebum production remained consistent from day 10 till day 25. Further analysis showed that 40% of participants had an overall reduction in pores by 50% or more, 10% had an overall reduction in the appearance of spots by 50% or more, 66% had an overall reduction in the amount of porphyrin in the skin by 50% or more and 55% had an overall increase in the amount of moisture present in the T-zone by 50% or more.

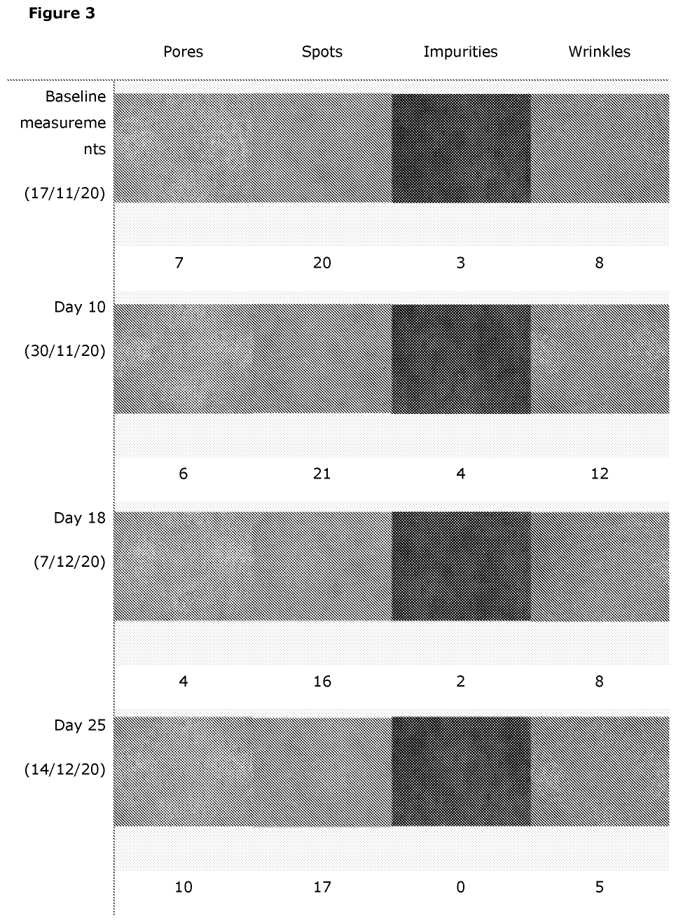

FIG. 3 shows observed results of a single participant from baseline to day 25 with the corresponding numerical value allocated by the skin analyser. This participant had an overall decrease from day 0 to day 25 in spots, impurities, and wrinkles. From day 0 to day 18 there was a consistent decrease in pores, yet on day 25 their result of 10 was higher than the baseline measurement of 7. The visual observations of the skin on day 25 show an improved skin texture and result as there is less shine and tighter looking pores.

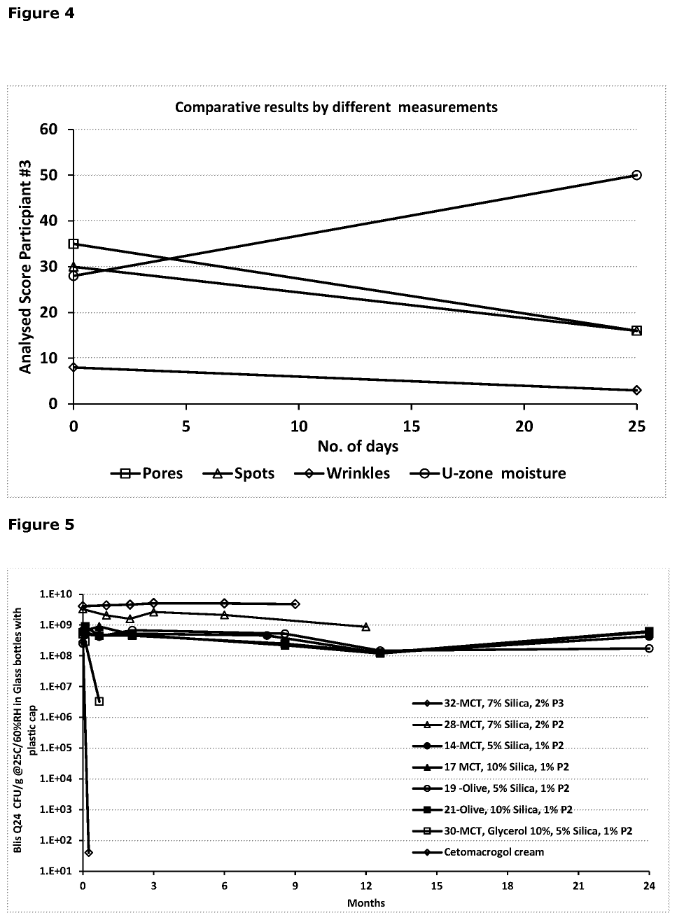

FIG. 4 shows example in reduction of scores for key parameters for a participant. Application of Blis Q24 moisturiser reduces the pore size, number of spots and wrinkles and enhances the hydration of the skin pre- and post-application.

### Example 6: Comparative Testing—Sedimentation and Caking

Three samples of grape seed oil suspension were made following the process described in WO 2006/104403: Example 3, using raw ingredient with three different particle sizes (Dv90: 290 μm, Dv90: 623 μm, Dv90: 1090 μm as measured by laser diffraction method of particle size analysis using dry powder dispersion (Malvern Instruments, USA).

Three versions of the serum formulation were made following the process described in Example 2 above using raw ingredient with three different particle sizes (Dv90: 290 μm, Dv90: 623 μm, Dv90: 1090 μm).

Q24 with a Dv90 of 290 μm was shown to pass through a 250 μm molecular sieve. Q24 with Dv90 of 623 μm or 1090 μm did not.

### Sedimentation

Two 10 ml syringes were filled with 6.4 mL of each of the six formulations, a 1 ml headspace gap was left for the grape seed oil suspensions to allow for mixing by shaking, and all syringes were sealed at the tip using blue tac.

The syringes containing the serum formulations were attached to the bench and the volume of sedimented Q24 raw ingredient was measured at time points 0, 0.8, 7, 15 and 30 days using the measurements on the syringes. The syringes containing the Grape seed oil suspensions were shaken, attached to the bench and the volume of sedimented Q24 raw ingredient was measured at time points 0, 1, 3, 5, 10, 30 and 60 minutes. Syringes of all formulations were left untouched, attached to the bench for 30 days.

The thickness of the Q24 Serum formulations resulted in no change in sedimentation volume with any Q24 raw ingredient particle sizes and visually the raw ingredient with a Dv90 of 290 μm resulted in the most even distribution. All the Grape seed oil suspensions had 100% sedimentation of the Q24 raw ingredient, with the largest particle size raw ingredient settling out the fastest within minutes.

### Caking

Once the sedimentation trial describes above was complete at 30 days, the grape seed oil suspensions were shaken vertically at a consistent pace with the Q24 raw ingredient ‘cake’ on the top side. Measurements of the raw ingredient remaining in the ‘cake’ unsuspended were taken every 10 seconds until 100% of the Q24 raw ingredient had been resuspended.

None of the serum formulations had any settling of Q24 and did not require testing for ‘caking’. All the grape seed oil suspension formulations had 100% settling of the Q24 raw ingredient and were tested. The grape seed oil suspensions made with Q24 raw ingredient with Dv90s of 290 μm and 623 μm resuspended 100% within 20 seconds, while the formulations made with Dv90 1090 μm raw ingredient took 40 seconds to resuspend 100%.

Therefore, the grape seed oil suspension described in WO2006/104403 did not show suitable sedimentation and anti-caking properties to be used as a topical cosmetic formulation according to the present invention.

### Example 7: Comparative Testing—Viscosity, Spreadability, and Stability

Four different formulations were prepared.
1. A grape seed oil suspension was made following the process described in WO2006/10440: Example 3, using Q24 raw ingredient with a Dv90 of 290 μm.
2. A deodorant stick formulation was made following the process described in WO2006/10440: Example 2, using Q24 raw ingredient with a Dv90 of 290 μm.
3. A Cetomacrogol cream formulation was made according to Australian New Zealand Clinical Trials Registry (ANZCTR) trial ACTRN12616000022460 by mixing Q24 raw ingredient (250pm sieved) killed by gamma irradiation into commercially purchased Cetomacrogol cream (HealthE Non-ionic cream, Jaychem, New Zealand).
4. A serum formulation BLT17Q24-31 was made according to Example 2, using Q24 raw ingredient with a Dv90 of 290 μm.

### Viscosity

Each of the formulations were filled into a 10 ml syringe, the bubbles tapped out and dispensed onto a glass microscope slide by pressing down the plunger. The ease or difficulty of dispensing was recorded, and the formulations visually assessed for suitability of thickness, Q24 raw ingredient distribution and likely ability to be dispensed evenly from the two most common forms of facial moisturiser packaging, pump bottles or small round containers for dipping fingers to collect formulation before application.

TABLE 3
Visual viscosity observations of Q24 formulations

| | | | 3: Cetomacrogol | |
|---|---|---|---|---|
| | 1: Grape Seed | 2: Deodorant | cream | 4: Serum |
| Sample | Oil suspension | Stick formulation | formulation | formulation |
| Ease of | Runs out with no | Full force required. | Low amount of force | Low amount of force |
| dispensing | force applied. | Will not dispense | required. | required |
| from | Likely to | from a pumping | Likely to dispense | Likely to dispense |
| syringe at | dispense | system or a small | consistent volumes | consistent volumes |
| room temp | consistent | container. | and consistent Q24 | and consistent Q24 |
| | volumes, but | | doses from a | doses from a |
| | inconsistent Q24 | | pumping system or | pumping system or |
| | doses due to | | small container. | small container. |
| | sedimentation | | | |
| | rate, from a | | | |
| | pumping system | | | |
| | or small | | | |
| | container. | | | |
| Visual | Thin, runny with | Thick, almost solid | Medium thickness | Medium thickness |
| thickness | non-homogenous | with homogenous | that settles when | that maintains |
| and Q24 | raw ingredient | raw ingredient | dispensed with | shape when |
| raw | distribution | distribution | homogenous raw | dispensed with |
| ingredient | | | ingredient | homogenous raw |
| distribution | | | distribution | ingredient |
| | | | | distribution |
| | | | | Does not form a |
| | | | | ‘skin’ when mixed |
| | | | | with hydration |
| | | | | moisturiser over |
| | | | | short exposure time |
| Feel on | Gritty due to | Solid to start, non | Non-gritty | Non-gritty with or |
| application | being non | gritty once melted | | without mixing with |
| | homegenous | | | hydration |
| | distribution of | | | moisturiser |
| | particles | | | |
| Suitability | Not suitable | Not suitable | Excellent | Excellent |
| for facial | | | | |
| skin | | | | |
| application | | | | |
| based on | | | | |
| visual | | | | |
| thickness | | | | |

Formulations 1 and 2 were not considered suitable for facial cosmetic skin application due to being too runny and too thick, respectively.

### Spreadability

To determine the mechanical spreadability of samples 1 and 4, a 2 cm diameter circle was marked with vivid onto the underside of a flat petri dish lid. Then 0.5 g of a formulation was placed on the top of the lid evenly covering the 2 cm diameter circle. A second petri facing top down with a 200 g mass was placed on top of the formulation and allowed to sit for 20 seconds. The diameter of the circle of spread formulation was then measured. The following equation was used to determine the percentage of spreadability. (Method: Saleh A. et. al, “Evaluation of Skin Permeation and Analgesic Activity Effects of Carbopol Lornoxicam Topical Gels Containing Penetration Enhancer”, The Scientific World Journal, vol. 2014, Article ID 127495, 9 pages, 201) was used to design a slightly modified test method suitable for the formats being tested.

To determine the consistency of microbial growth once spread, 0.1 g of each formulation was placed on the top of a human blood agar plate. A swab was then used to spread the formulation evenly down the plate in 14 left to right streaks. The thickness of the deostick did not allow for even spreading down the agar plate, and a second method was used where the formulation was pre-melted and then spread down the plate.

Formulation 4 showed excellent physical and microbial spreadability compared to Formulation 1, suitable for body/facial skin application.

### Stability

Formulation 3 was prepared by weighing 19.8 g of Cetomacrogol 100BP cream (HealthE, Non-ionic cream, B62236, Jaychem, New Zealand) into a stomacher bag using a sterile spoon. 0.2 g (2% in final formulation) of Blis Q24 raw ingredient was added to the Cetomacrogol cream, sealed and hand mixed until a homogeneous cream was formed. The cream was then placed in a stomacher and mixed for an additional 5 minutes. The Cetomacrogol Q24 cream was then dispensed into 30 ml glass vials, enumerated in triplicate. In a microcentrifuge tube (Eppendorf, USA), 0.1 g of M. luteus Q24 serum or cream was weighed and diluted with 0.9 g of warm (37° C.) Phosphate buffered saline and Polysorbate 80 (0.1% w/w). The mixture was homogenised by shaking using a vortex mixer (auto vortex) at 2800 rpm for 5 min under ambient conditions (20° C.±2° C.) to obtain a homogeneous dispersion. 100 μL. was then appropriately serially diluted with PBS, spread plated onto hBaCa agar plate and incubated at 37° C./5% CO₂ for 28 h-48 h. The colonies were counted and calculated for bacteria concentrations in CFU/g, using a Q-Count Automatic Colony Counter (Spiral Biotech, New Zealand). The vials were then placed into an incubator set at 25° C./60% RH for stability testing at time points 7, and 14 days.

Formulation 4 was made following the process described in Example 2. The serums were then dispensed into 30 ml glass vials, enumerated in triplicate. The vials were then placed into an incubator set at 25° C./60% RH for stability testing.

Blis Q24 was found to be highly unstable in Formulation 3. A 7-log drop was observed after just 7 days and no viable cell count was found at 14 days when stored at 25° C./60% RH. In comparison Blis Q24 was very stable for at least 24 months at 25° C./60% RH with varying silica contents, Q24 percentages and in MCT and olive oils (FIG. 5).

### Example 8: Comparative Sensory Testing

Formulations 1, 2, and 4 were prepared as described in Example 7. Formulation 5 was prepared by providing the serum formulation 4 in a dual chamber bottle with a hydration moisturizer as described herein.

Each formulation was set-up in a clean beaker and placed on a labelled white background. Sterile swabs were provided for each participant to gather and dose the formulations. Each participant (5 female and 3 male (n=8)) was provided with a score sheet with descriptions of how to test for each sensory property and the scoring system. Each were asked to follow the instructions provided to assess and score each formulation on each sensory property individually.

The properties assessed were:
Consistency
Cushion effect
Distribution (spreading)
Tackiness/stickiness
Absorption rate
Hydration

Participants were also asked to rate their choice of formulation based on appearance, preference, applicability, whether it was make-up friendly and whether they would recommend to others.

Formulations 3 and 5 scored significantly higher than formulations 1 and 2 for all parameters tested.

### Example 9: Safety and Efficacy

The safety and efficacy of Blis Q24 bacterial cells was evaluated using the EpiDerm 3D Skin Model.

### Methodology

### EpiDerm Human 3D Skin Model Culture (Skin Model)

Upon receipt, the final development of the immature EpiDerm (EPI-201-4D; MatTekCorporation, Ashland, MA, USA) tissues was completed following the manufacturer's instructions. In brief, the tissues were transferred from the agarose-containing gel on which they were shipped to cell culture plates containing pre-warmed differentiation medium (EPI-201-DM). They were then maintained at 37° C. in a 5% CO2 atmosphere for 4 days, with 2 or a changes to fresh differentiation medium during that period. Finally, the fully developed EpiDerm tissues were transferred to fresh plates containing standard culture medium (EPI-100-NMM) for use in each experiment.

### Dose Response Experiment for Tolerability

The maturation of 24 immature EpiDerm tissues was performed as described above. The fully differentiated tissues were randomly assigned to receive either PBS(negative control) or Blis Q24 at one of 5 dose levels (1×10⁵ cfu/ml to 1×10⁹ cfu/ml), with 4 replicate tissues used per treatment. The apical surfaces of the tissues were treated with 30 μL of the treatment solutions, and the tissues were then cultured for 24 h at 37° C. in a 5% CO₂ atmosphere. At the end of that period the tissues were gently rinsed with PBS and their viabilities were assessed using the MTT viability assay (Mosmann, T. (1983). Journal of Immunological Methods, 65(1-2), 55-63, Kubilus, J. (1996). In Vitro and Molecular Toxicology, 9(2), 157-166.).

### Safety and Efficacy Experiment

The maturation of 36 immature EpiDerm tissues was performed as described above. The fully differentiated tissues were numbered from 1-36 and were randomly assigned to receive one of the treatments shown in Table 3. This was done such that each of the 6 combinations of main treatment (PBS, Q24 or vitamin C) and 0.5% SDS exposure treatment (±0.5% SDS) was randomly represented on each of five 6-well cell culture plates. The remaining 6 tissues, which were destined for histology or else to receive 5% SDS, were grouped together on the sixth cell culture plate. Following maturation, the 36 tissues were cultured in the assay medium for 24 h at 37° C. in a 5% CO₂ atmosphere, after which the baseline (day 0) conditioned media samples were collected and stored frozen at −80° C. for cytokine measurement. For 4 days starting from day 0, each tissue was treated daily with the appropriate main treatment solution before the tissues were returned to the incubator maintained at 37° C. with a 5% CO2 atmosphere. Tissues that were treated with Blis Q24 or the PBS vehicle had 30 μL of the appropriate solution applied to the apical surfaces of the tissues, after the remnants of the previous day's doses were rinsed off with PBS. Vitamin C (50 μg/mL) was instead delivered in the culture medium. On day 4 conditioned media samples were collected and stored frozen at −80° C. for cytokine measurement. 33 of the tissues were then exposed for 30 μL of 0.5% or 5% SDS in PBS, or to PBS as negative control. After 1 h these tissues were rinsed with PBS and dabbed dry with cotton buds before re-application of the appropriate main treatment (PBS, Blis Q24 or vitamin C) and transferral to plates containing fresh medium. The remaining 3 tissues (destined for histology) were simply fed fresh medium and given the appropriate main treatment. All tissues were returned to the incubator for a further 24 h at 37° C. in a 5% CO2 atmosphere, after which time the day 5 conditioned media were collected and stored frozen at −80° C. All tissues were gently rinsed with PBS and the 3 histology tissues were fixed in neutral buffered formalin. The viabilities of the other 33 tissues were assessed using the MTT viability assay.

### MTT Viability Assay (Safety)

EpiDerm tissues on cell culture inserts were added to 6-well cell culture plates containing 0.9 mL of 1 mg/mL MTT in DMEM medium (Thermo Fisher Scientific) and were cultured for 3 h at 37° C. in a 5% CO₂ atmosphere. They were then rinsed with PBS and were blotted dry on cotton gauze. Moisture remaining on the insides of the inserts was removed by use of cotton buds, after which each insert and tissue was immersed in 6 mL of propan-2-ol and soaked overnight in the dark in a sealed 20 mL scintillation vial. The resultant purple solutions of formazan dye were transferred to disposable cuvettes and their absorbances at 570 nm and 650 nm (reference wavelength) were measured in an Ultrospec UV-visible spectrophotometer (LKB). Data analysis involved subtraction of the absorbance at the reference wavelength from the 570 nm reading to give corrected absorbance values. These were converted to % viability values by dividing each absorbance value by the mean of the negative control group (PBS without exposure to 0.5% SDS) and multiplying by 100.

### Histology (Efficacy)

Tissues were fixed in neutral-buffered formalin (LabServ) and were sent to Gribbles Veterinary Lab (Christchurch, NZ) for preparation of H&E-stained sections. Light microscopy was performed on a Leica DM6000 B microscope (Leica Microsystems, Switzerland) and images were acquired using Leica Application Suite v4.12 software.

### Cytokine Quantification for Anti-Inflammatory Effect (Efficacy)

For quantification of IL-1β, IFN-α2, IFN-γ, TNF-α, MCP-1 (CCL2), IL-6, IL-8 (CXCL8), IL-10, IL-12p70, IL-17A, 1L-18, IL-23, and IL-33, bead-based multiplex LEGENDplex™ analysis(LEGENDplex™ Human Inflammation Panel 1 (13-plea); BioLegend, San Diego, CA, USA) was used according to the manufacturer's instructions. The relevance of each of the measured cytokines is described in Table 4. Reactions were performed in duplicate. Analysis was performed with the Cytek™ Aurora flow cytometer (Cytek Biosciences Inc., Fremont, CA, USA). Data were analysed via Legendplex V8.0 software (BioLegend) and specified as pg/mL.

### Statistical Analysis

Statistical analysis and graphing were performed using Prism 9.2.0 (GraphPad Software). Data were analysed by one-way or two-way analysis of variance (ANOVA), with correction for multiple comparisons using Dunnett's or Šidák's method, as appropriate.

### Tolerability

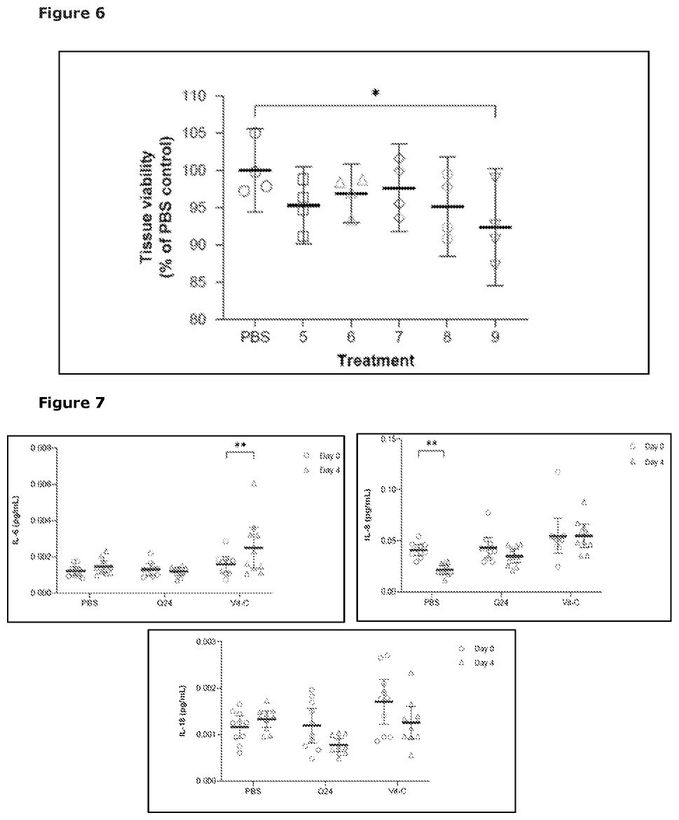

Tolerability of Blis Q24 was determined via dose response experiment. EpiDerm tissues treated with live Blis Q24 (1.42×10⁵, 1.31×10⁶, 1.46×10⁷, 1.71×10⁸ and 1.63×10⁹ cfu/ml) suspension in phosphate-buffered saline (PBS) showed >90% viability (FIG. 6). Although viability for 1×10⁹ CFU/ml was significantly different from PBS control, the mean % cells were still >90% viable indicating that Blis Q24 was well tolerated by the model skin tissues during the 24 h exposure.

These in vitro results compare well with the Blis Q24 Hydration serum in vivo trials where no skin irritation was observed even after repeated dosing with 1.5×10⁸ CFU/dose.

Conclusion: Blis Q24 in different clinically relevant doses is well tolerated in tissue culture model indicating safety of the strain and the products containing Blis Q24.

### Safety

Following the dose response experiment a dose of 1×10⁸ CFU/ml was selected for safety and efficacy studies.

Measurement of 13 cytokines (day 0 and 4) was carried out in EpiDerm tissues treated with Blis Q24 (1×10⁸ CFU/ml), PBS (negative control and Vit C (positive control). Out of 13 treatment related effect was observed only 3 cytokines. The interleukin-6 (IL-6), interleukin-8 (IL-8) and interleukin-18 (IL-18) are pro inflammatory cytokines known to be produced by the skin cells in response to the inflammatory stimuli. IL-6 levels showed a slight but significant increase on day 4 for vitamin C but were unchanged between the two days for Blis Q24 and PBS (FIG. 7 top left). IL-8 levels were slightly lower on day 4 for Blis Q24 and PBS (although the change was only significant for the latter), and unchanged for vitamin C (FIG. 7 top right). For IL-18, there were no significant differences in levels on days 0 and 4, although Blis Q24 and vitamin C showed a trend for lower values on day 4 (FIG. 7 bottom middle).

Conclusion: The results indicate that Blis Q24 is safe as it did not elicit in an inflammatory immune response in Epiderm tissues following daily administration for 4 days.

### Efficacy (Histology)

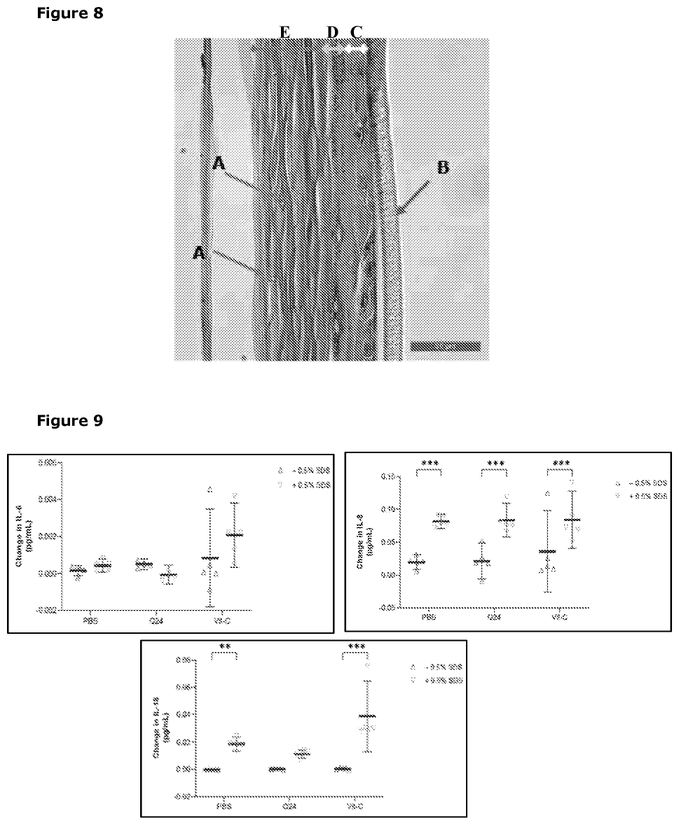

A dose of 1×10⁸ CFU/ml of live Blis Q24 suspension in PBS, PBS (negative control) and Vit C (positive control) was applied to the EpiDerm cells (duration 4 days) and treatment related changes to tissue morphology were recorded (n=1, FIG. 8). Although, no obvious treatment-related differences were apparent, the tissue that received Blis Q24 revealed nuclear remnants caught in the stratum corneum which indicates possible acceleration of stratum corneum production. The stratum corneum was also slightly thicker in the Blis Q24 tissue than in those treated with PBS or vitamin C. More replicates are required to confirm these findings but there seem to be a positive effect of Blis Q24. It is known that the stratum corneum primarily functions as a barrier between the deeper layers of skin and the outside environment, preventing toxins and bacteria from entering the body. It also helps to keep moisture from evaporating into the atmosphere and so is important for keeping the skin hydrated.

This result indicates that Blis Q24 may help in rejuvenating and hydrating the skin by accelerating the formation of stratum corneum.

Conclusion: Blis Q24 is effective in accelerating the growth of stratum corneum responsible for barrier function and moisture retention. These in vitro results substantiate the findings of in vivo trials where increase in hydration and reduction in pores and wrinkles observed by the study participants upon application of Blis Q24 hydration serum.

### Anti-Inflammatory Effect

Only IL-6, IL-8 and IL-18 had detectable levels to determine the effect of treatment Blis Q24, PBS (negative control and Vit C positive control) (FIG. 9—top left).

Pre-treatment with Blis Q24 caused a slight nonsignificant reduction in the amount of IL-6 released in response to 0.5% sodium dodecyl sulphate (SDS), in contrast, the vitamin C-treated tissues displayed a slight increase on day 5 in response to the 0.5% SDS exposure. Significant increases in IL-8 levels were evident on day 5 compared to day 4 in response to 0.5% SDS for all three main treatment groups (FIG. 14—top right). IL-18 release was also stimulated by 0.5% SDS in all groups, but the change was not significant for the Blis Q24 group (FIG. 14—bottom middle).

Conclusion: The slight reduction in the SDS-induced release of IL-6 and the blunted increase in IL-18 for the Blis Q24+0.5% SDS group, indicates the anti-inflammatory effect of Blis Q24.

### Example 10: Placebo Trials

Two trials were conducted to determine the effect of the inventive formulation on cosmetic skin parameters.
Trial 1: Active Q24 serum (oil phase)+aqueous phase vs Placebo serum+aqueous phase.
Trial 2: Live Q24+Cetomacrogol v Dead Q24+Cetomacrogol.

Trial 1: In this randomized, placebo controlled, double blind trial, 8 participants were divided into two groups. Group 1 (n=4) (active group) were given a dual chamber bottle in which chamber 1 consisted of serum containing active Blis Q24 (1×10⁹ cfu/g) and chamber 2 contains aqueous phase. Group 2 (n=4) (placebo group) were also given a dual chamber bottle in which chamber 1 consisted of serum base (placebo without Blis Q24) and chamber 2 contains aqueous phase. One participant in Group 1 didn't complete the trial due to reasons not related to trial.

Trial 2: In this randomized controlled double blind trial, 8 participants were divided into two groups. Group 1 (n=4) (live Q24 group) were given a dual chamber bottle in which chamber 1 consisted of serum containing live Blis Q24 (1×10⁹ cfu/g) and chamber 2 contains Cetomacrogol cream. Group 2 (n =4) (Dead Q24 group) were also given a dual chamber bottle in which chamber 1 consisted of serum containing gamma irradiated Blis Q24 (Dead) and chamber 2 contains Cetomacrogol cream.

Participants in both trials were then asked to pump out the formulation (1 pump each of serum and moisturiser or cream) mix them on the palm and apply on the face twice daily (morning and night) for 31 days.

Skin quality parameter analysis of the full face was carried out using an advanced skin analyser device (Chowis, South Korea) before the first application (Day 0 pre trial) on day 11 and day 31 during the trial and 10 days post last application (day 41).

### Trial 1: Active vs Placebo

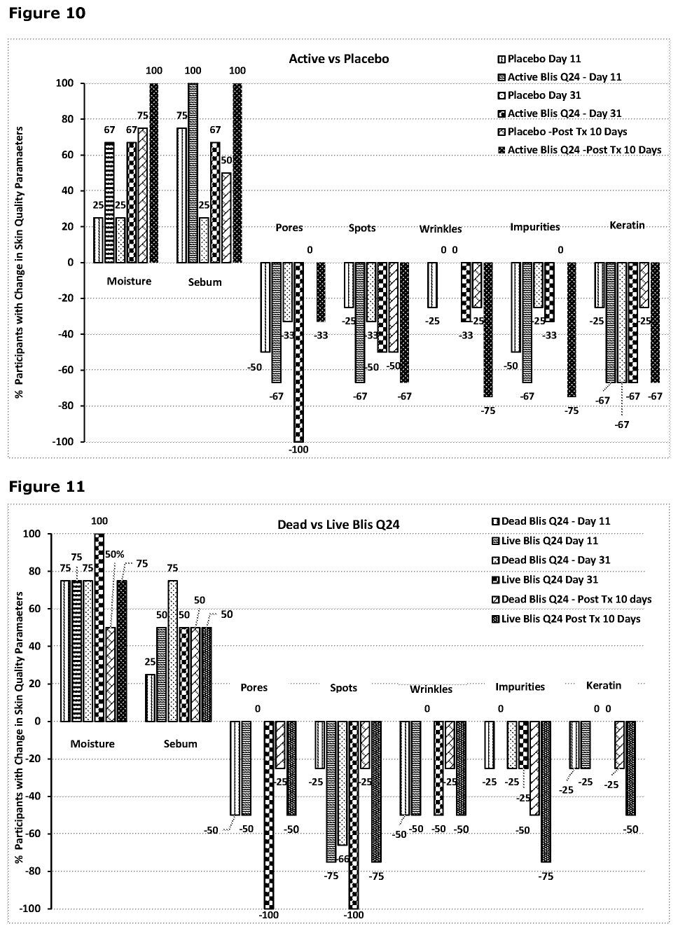

Blis Q24 “active” composition showed improvement in all skin quality parameters at various time points versus Placebo compared to baseline (FIG. 10). The improvement was visible even after 10 days post treatment (Tx).

In this study an increase in sebum was also observed. The interpretation of sebum score is difficult as sebum plays an important multi-faceted role: Sebum lubricates the skin to protect against friction and makes it more impervious to moisture. It reduces water loss from the skin surface. It protects the skin from infection by bacteria and fungi. Furthermore, the sebaceous gland transports antioxidants in and on the skin and exhibits a natural light protective activity. An overproduction of sebum can lead to oily skin.

Note in the present study none of the participant complained of “oily skin” suggesting the level of sebum was well within the desired for the participant with all showing an increase in sebum and moisture content. Increase in sebum was also observed in placebo group. Therefore, an increase or decrease in sebum compared to baseline is not necessarily “desired” or “undesired”, rather the balance of sebum is the important factor.

### Trial 2: Live Q24 vs Dead

Blis Q24 “Live” composition showed improvement in all Skin quality parameters at various time points versus Dead compared to baseline (FIG. 11). The improvement was visible even after 10 days post treatment (Tx).

### Example 11: Addition of Prebiotics

### Inhibition of Common Prebiotics

CABK12 agar plates were split into segments of 6 and lawned with a suspension of M. luteus Q24 raw ingredient P3. Each of the aqueous potential prebiotic substances to be screened were serially diluted in a range of concentrations from 100% to 0.3% using sterile distilled water. Oil based substances were tested at 100% only.

20 μL of each concentration, of each substance were pipetted into a spot onto one segment of the lawned CABK12 agar plate and was incubated for 24 hours at 37° C. 5% CO₂ in air.

TABLE 8
Summary of prebiotic MIC results vs Blis Q24 and the
prebiotic potential uses based on inhibitory concentrations
Potential Prebiotics

| | Suitable to be used in | |
|---|---|---|
| | Q24 containing | |
| Ingredient | serum/product? | Appearance |
| Allantoin | Yes | Powder |
| Aloe Vera Powder | Yes | Powder |
| Colloidal Oatmeal USP organic | Yes | Powder |
| Xylitol | Yes | Powder |
| Yeast Extract | Yes | Powder |
| Fructo-oligosaccharide (FOS)- | Yes | Powder |
| Powder | | |
| Fructo-oligosaccharide (FOS)-Liquid | Yes | Oil |
| Manuka Honey Powder | Yes | Powder |
| Niacinamide | Yes | Powder |
| Green Tea Powder | No (inhibitory at all | Powder |
| | concentrations) | |
| Flaxseed Oil | Yes | Oil |
| Olive Squalene | Yes | Oil |
| Pomegranate Seed Oil-Cold pressed | Yes | Oil |
| Vitamin E, D Alpha Tocopherol | Yes | Oil |
| 1000IU | | |
| Retinol (Vitamin A) | Yes | Oil |
| Vitamin C (Ascorbic acid) | Yes | Powder |
| Hyaluronic acid | Yes | Powder |
| Salicylic acid (SA) | No (inhibitory at >5%) | Powder |
| Lactic acid (LA) | No (inhibitory at >2.5%) | Powder |
| Calendula oil | Yes | Oil |
| Almond oil | Yes | Oil |
| Tomato oil | Yes | Oil |
| Sunscreen SPF (Neutrogena Ultra | Yes | Powder (in aqueous |
| sheer face and body) | | cream) |

Conclusion: All substances passed the first screening test for a non-Q24 containing product except green tea powder which was inhibitory at all concentrations tested. 6 other potential prebiotics showed some inhibitory effect on Q24 indicating they would not be suitable for use in a product requiring Live Q24 stability, but maybe of use in a prebiotic only formulation.

### Effect of Prebiotics on the Growth of Blis Q24

A batch of M17 broth (Difco #218561) was made, excluding the lactose solution, by following the manufacturer's instructions. 50 ml of broth was dispensed into sterile 100 ml Schott bottles and 2.5 g (5%) of each potential prebiotic substance was added and mixed well using a magnetic stirrer and stir plate. The mixtures were autoclaved at 110° C. for 10 minutes and allowed to cool.

The suspensions of the prebiotic candidates were prewarmed to 40° C. in order to allow for increased homogenisation of any oil-based components prior to dispensing into the wells. 2 ml of each suspension and an M17 only (control) were pipetted into a sterile 24 well tissue culture plate. A suspension of Q24 raw ingredient P3 was made in PBS and adjusted to an optical density of 0.125. 100 μl of the suspension was pipetted into each well. Once the suspension was added each well was mixed by aspirating and dispensing the solution 5 times with a 1 ml pipette.

Each sample was enumerated at time points 0, 3, 6, 15, 26 and 34 hours using the following method. At each time point the wells were mixed by using a 1 ml pipette and dispensing and aspirating 5 times. Then 100 μl was removed from each well and added to a 900 μl Eppendorf of PBS ( 1/10 dilution). These 1/10 dilutions were vortexed horizontally for 10 minutes at 2600 rpm. Then the samples were serially diluted by transferring 100 μl of the 1/10 dilution into a fresh 900 μl PBS, this was repeated until serially diluted 6 times. 20 μl of each dilution from each sample was spotted onto a sheep blood agar plate in triplicate. The spots were left for 30 minutes to dry and placed into a 37 C 5% CO₂ incubator for 28-36 hours. The number of colonies in each spot were then counted using an electronic colony counter and average to give the final result.

Results: All the prebiotic candidates tested in showed an increase in Q24 higher than that of the control with most resulting in a 1 to 2-log increase over 24 hours compared to the control.

Conclusion: Surprisingly commonly used prebiotics (carbohydrates (except for Oatmeal)) typical for topical application have not supported growth of Q24.

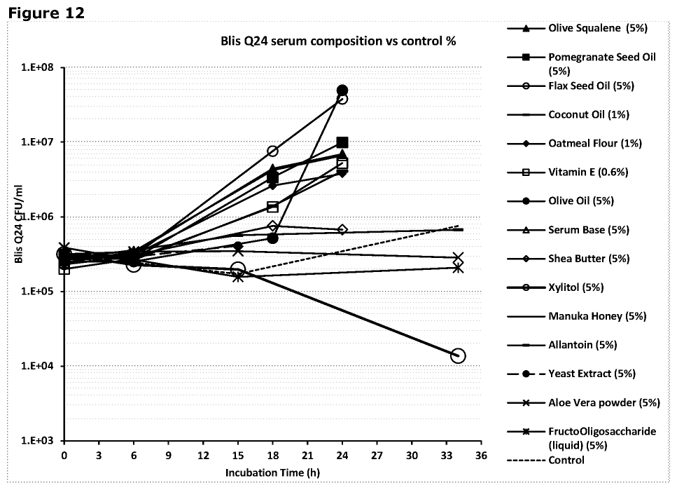

Conventional substances used as prebiotic for gut bacteria did not perform well at increasing the growth of Q24, some such as xylitol and Manuka honey were detrimental to the live Q24 cell count at 24 hours (FIG. 12).

In contrast oil-based substances typically used as functional actives and other cosmetic ingredients performed very well, with the best increasing the live cell count of Q24 by 2-log over 24 hours.

### Combinations of Prebiotics

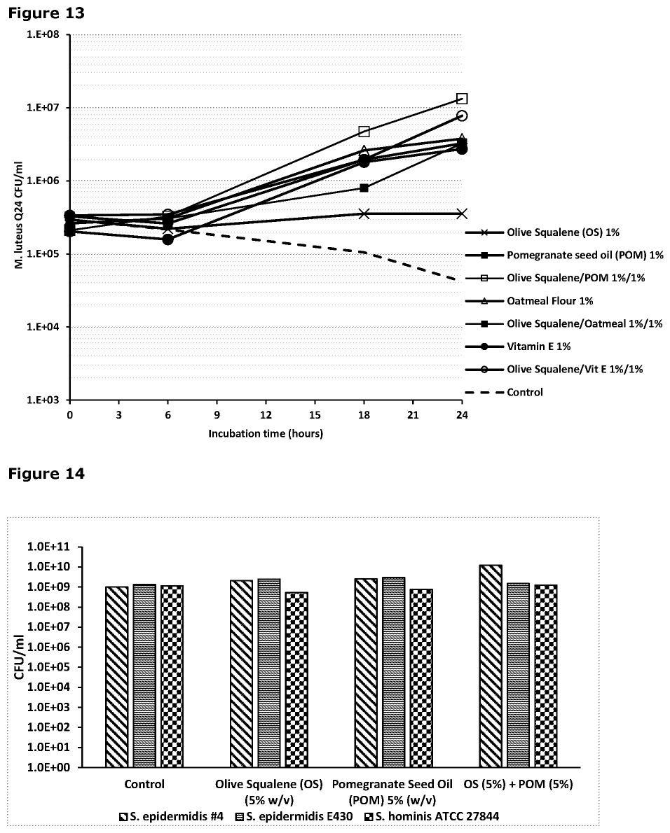

The same method was used as in research question 10 except the time points tested were 0, 6, 18 and 24 hours. Olive squalene was selected as one of the preferred prebiotics with 3 others that were analysed in combination with olive squalene and compared to the results of the prebiotic candidates alone (FIG. 13).

Results: The growth of the Q24 was increased well above the sum of olive squalene and pomegranate seed oil separately, showing a synergistic response when both prebiotics are together resulting in an increased growth rate of Q24.

The growth of the Q24 was the same as the sum of olive squalene and oatmeal flour (colloidal oatmeal) separately, showing no synergistic response for this combination.

The growth of the Q24 was increased well above the sum of olive squalene and vitamin E separately, showing a synergistic response when both prebiotics are together resulting in an increased growth rate of Q24.

Conclusion: Both olive squalene+pomegranate seed oil and olive squalene+vitamin E were found to have a synergistic relationship increasing the growth rate of Q24 compared to either alone. Olive squalene and oatmeal flour did not show this same response. A formulation made to include both olive squalene and either pomegranate seed oil or vitamin E will be more efficacious than a formulation including only olive squalene, pomegranate seed oil, or vitamin E.

### Prebiotic Activity Against Some Skin Commensals

The same method was used as above for Blis Q24 ingredient except the suspensions with optical density of 0.125 were prepared for each of the 3 selected skin commensals.

The skin commensals selected were:
S. epidermidis #4—Sensitive to Q24 inhibition
S. epidermidis E30—Resistant to Q24 inhibition
S. hominis ATCC 27844—Mixed resistance condition dependent

S. hominis ATCC 27844 is available from American Type Culture Collection (ATCC); S. epidermidis #4 and S. epidermidis E30 are available from BLIS Technologies Ltd on request.

Results: No additional growth advantage observed with or without prebiotics for skin commensals, suggesting that prebiotics selectively promote the growth of Blis Q24 (FIG. 14).

Conclusion: Olive Squalene and Pomegranate seed oil on their own or in combination do not offer any advantage compared to control media for skin commensals, an effect more pronounced and specific for Blis Q24.

1. A method to improve the appearance of skin or at least one sign of aging comprising applying to the skin a topical composition comprising Micrococcus luteus Q24.

2. A method according to claim 1, wherein the composition comprises Micrococcus luteus Q24 in an amount of about 1×10³ to about 1×10¹² cfu/g.

3. A method according to claim 1, wherein the composition comprises a viscosity modifier.

4. A method according to claim 3, wherein the composition comprises the viscosity modifier in an amount of about 3 to about 15% w/w.

5. (canceled)

6. A method according to claim 1, wherein the composition comprises a dispersing agent.

7. A method according to claim 6, wherein the composition comprises the dispersing agent in an amount of about 0.1 to about 5% w/w.

8. (canceled)

9. A method according to claim 1, wherein the composition comprises an oil vehicle.

10-13. (canceled)

14. A method according to claim 1, wherein the composition further comprises one or more additional probiotics.

15. A method according to claim 14, wherein the one or more additional probiotic is selected from the group consisting of a Streptococcus spp., a Lactobacillus spp., Limosilactobacillus spp., a Lacticaseibacillus spp., a Ligilactobacillus spp., Lactiplantibacillus spp., a Bifidobacterium spp., a Saccharomyces spp., and a combination of any two or more thereof.

16-17. (canceled)

18. A method according to claim 1, wherein the composition further comprises an inhibitory activity enhancer, a buffering agent, an antibacterial agent, a prebiotic, a fragrance, an antioxidant, a colourant, a skin protective agent, an antimicrobial, an aluminium salt, a mineral pigment, an odour absorbent or neutraliser, a sunscreen agent, or a combination of any two or more thereof.

19-26. (canceled)

27. A method according to claim 1, wherein the composition comprises
a) from about 0.1% to about 10% w/w olive squalene, and
b) from about 0.1% to about 10% w/w pomegranate seed oil, or from about 0.1% to about 3% w/w vitamin E.

28. (canceled)

29. A method according to claim 1, wherein improving the appearance of skin or at least one sign of aging includes that skin looks more radiant, skin looks healthier, skin feels more hydrated, pores are reduced in size, skin feels softer, skin looks clearer, wrinkles are reduced, dryness is reduced, spots are reduced, impurities are reduced, moisture is increased, or sebum production is decreased.

30. A method according to claim 1, wherein the composition is non-aqueous.

31. A method according to claim 1, wherein when applied, the composition is applied in combination with an aqueous phase.

32. (canceled)

33. A method according to claim 1, wherein the composition has a shelf-life of at least 6 months at 25° C. at 60% RH.

34. A topical composition comprising Micrococcus luteus Q24, a viscosity modifier, a dispersing agent and an oil vehicle, wherein the composition comprises Micrococcus luteus Q24 in an amount of about 1×10⁴ to about 1×10¹⁰ cfu/g.

35. (canceled)

36. A topical composition according to claim 34, wherein the viscosity modifier is hydrophobic silica and the dispersing agent is polysorbate 80, and wherein the composition comprises
about 2% to about 10% w/w hydrophobic silica,
about 0.5% to about 2% w/w polysorbate 80, and
a quantity sufficient amount of oil vehicle.

37. A topical composition according to claim 36 wherein the oil vehicle is a medium chain triglyceride.

38. A topical composition according claim 36, further comprising 0.1% to 35% w/w prebiotic(s).

39. A topical composition according to claim 38 wherein the prebiotic(s) is selected from olive squalene, pomegranate seed oil, vitamin E, or a combination thereof.

40-49. (canceled)
